(19) United States
(12) Patent Application Publication
(10) Pub. No.: US 2025/0281485 A1
(43) Pub. Date: Sep. 11, 2025

(54) DIAGNOSIS AND/OR TREATMENT OF HEART FAILURE WITH PRESERVED EJECTION FRACTION

(71) Applicants: UNIVERSITEIT MAASTRICHT, Maastricht (NL); ACADEMISCH ZIEKENHUIS MAASTRICHT, Maastricht (NL)

(72) Inventors: Harald Horst Heinz Wilhelm SCHMIDT, Aachen (DE); Cristian Nogales CALVO, Maastricht (NL); Alexandra PETRAINA, Maastricht (NL); Kai KNÖPP, Halle (Saale) (DE); Hermann Alois Martin MUCKE, Vienna (AT); Daniel Gerhard Franziskus SEDDING, Hannover (DE)

(73) Assignees: UNIVERSITEIT MAASTRICHT, Maastricht (NL); ACADEMISCH ZIEKENHUIS MAASTRICHT, Maastricht (NL)

(21) Appl. No.: 18/857,198

(22) PCT Filed: Mar. 23, 2023
(86) PCT No.: PCT/NL2023/050148

§ 371 Date: Oct. 16, 2024

(30) Foreign Application Priority Data
Apr. 22, 2022 (NL) 2031662

(51) Int. Cl. A61K 31/506; A61K 31/198; A61K 31/519; A61P 9/04; G01N 33/573

(52) U.S. Cl. CPC A61K 31/506; A61K 31/198; A61K 31/519; A61P 9/04; G01N 33/573; G01N 2333/90209; G01N 2800/325

(57) ABSTRACT

The present invention relates to compositions and methods for treating and/or preventing heart failure with preserved ejection fraction (HFpEF), especially in subjects with an impaired NO-cGMP-PKG signalling axis. The inventors established that not all patients with clinically defined HFpEF suffer from insufficient NO-cGMP-PKG signalling. In accordance with the invention, patients with an HFpEF diagnosis can be selected/stratified based on biomarkers such as NADPH oxidase Type 5 (Nox5) plasma levels, nitration of tyrosine residues on plasma proteins and/or cell-based assays. Moreover, the inventors have found that treating this signalling network at two or more nodes, especially sGC and NO synthase, achieves the first-in-class mechanism-based, causal and high precision therapy for HFpEF endotype which is defined by insufficient NO-cGMP-PKG signalling.

### FIELD OF THE INVENTION

The present invention relates to compositions and methods for treating and/or preventing heart failure with preserved ejection fraction (HFpEF), especially in subjects with an impaired NO-cGMP-PKG signalling axis. The present invention provides methods of treating such subjects, which methods may include steps of assessing whether the subject is eligible for the treatment, and compositions for use in said treatments.

### BACKGROUND OF THE INVENTION

Cardiovascular diseases cause by far the most significant burden of disease worldwide, as measured in Disability-Adjusted Life Years (DALYs). Its most substantial fraction comes from chronic heart failure, an umbrella term for a complex set of conditions that permanently reduce the heart muscle's efficiency to pump or fill with blood. The prevalence of diagnosed heart failure in developed countries is generally estimated at 1-2% of the general adult population; however, about half of all cases might go undetected. In those aged 65 years and over, the actual all-type prevalence has been estimated at 11.8% in developed countries. Worldwide there might be over 65 million people affected at any time.

There are two major subgroups of chronic heart failure. One, originally known as systolic heart failure and now termed heart failure with reduced ejection fraction (HFrEF), is characterised by the fact that the percentage of the volume of blood ejected from the left ventricle with each heartbeat divided by the maximal filling capacity is significantly below the norm. In the other form, HFpEF (earlier known as diastolic heart failure), the ejection fraction is relatively preserved, but the ventricle is too stiff to relax during diastole properly and therefore is improperly filled. This ventricular stiffness usually develops against a background of old age, diabetes, and fibrotic conditions. Myocardial remodelling in HFpEF differs from HFrEF, in which remodelling is driven by the loss of cardiomyocytes. The cellular, molecular, and metabolic differences between HFrEF and HFpEF are more pronounced than their advanced clinical symptoms.

Once considered relatively rare, HFpEF is now known to affect at least half of all heart failure patients and is responsible for the majority of heart failure cases in older diabetics. Temporal trends show that an increasing incidence of HFpEF balances the decreasing incidence of HFrEF over the last two decades. The epidemiological dynamics might be even worse because HFpEF patients may not have significant symptoms at rest in the earlier stages of the disease, showing only increasing exercise intolerance, and might remain undiagnosed for longer. While the reversal of cardiac remodelling can be achieved, and cardiac function improved, in HFrEF by treatment with angiotensin-converting enzyme inhibitors and β-blockers, HFpEF remains without specific pharmacological options beyond therapy for the underlying risk factors, despite many clinical trials that have been conducted using widely differing strategies.

The mechanisms that drive the development and maintenance of HFpEF are still far from clear, which is a significant cause for the lack of effective pharmacological treatments. Moreover, as the present inventors have found, clinical HFpEF is not a molecularly monolithic disease but rather the common final endpoint of different endotypes, developing along different pathways.

In 2013 Paulus and Tschöpe (J Am Coll Cardiol. 2013; 62 (4): 263-71) postulated a new paradigm for HFpEF. According to these authors, HFpEF develops based on a systemic proinflammatory state which causes coronary microvascular endothelial inflammation; this reduces nitric oxide (NO) bioavailability, cyclic guanosine monophosphate (cGMP) content, and ultimately activity of protein kinase G (PKG) in cardiomyocytes. The resultant hypophosphorylation of titin (a “molecular spring” responsible for the passive elasticity of muscle) would result in stiff cardiomyocytes and interstitial fibrosis. In this scenario of impaired NO-cGMP-PKG signalling, which is confirmed to exist in heart disease (J Cardiovasc Pharmacol. 2020; 75 (5): 370-84), HFpEF should, at least in theory, be treatable by restoring the activity of this axis. However, all attempts to accomplish this so far have failed. When the INDIE-HFpEF trial, investigating inorganic nitrite (JAMA 2018; 320 (17): 1764-73), and NEAT-HFpEF, which used organic nitrates (Am J Cardiol. 2019; 123 (10): 1660-66), had both failed to provide patient benefit, the new class of soluble guanylate cyclase (sGC) modulators moved into focus. Acting as stimulators and activators, these agents increased the enzymatic activity of sGC to generate cGMP independently of exogenous NO donors and had shown good efficacy in HFrEF. Praliciguat however failed in the CAPACITY-HFpEF trial (JAMA. 2020; 324 (15): 1522-1531) and so did vericiguat, in the VITALITY-HFpEF Phase III trial (JAMA. 2020; 324 (15): 1512-21), even though the Phase II SOCRATES-PRESERVED trial had raised hopes (Eur J Heart Fail. 2017; 19 (6): 782-91). All of this replicates the failure of the early RELAX study which pursued a variant of this strategy with the phosphodiesterase-5 (PDE5) inhibitor sildenafil. (JAMA. 2013; 309 (12): 1268-77)

It follows from the above that HFpEF continues to be a massive area of unmet medical need that will worsen, driven by demography and increasing prevalence of diabetes. It is the objective of the present invention to provide effective pharmacological treatments of HFpEF.

### SUMMARY OF THE INVENTION

The present inventors have surprisingly found that not all patients with clinically defined HFpEF suffer from insufficient NO-cGMP-PKG signalling, which has been considered a pervasive feature not only of heart failure as a whole but also of HFpEF. This previously unrecognized pathway heterogeneity will inevitably dilute the therapeutic effect if it is not considered, which provides at least a partial explanation for failure of clinical trials assuming a consistently impaired NO-cGMP-PKG signalling axis. The inventors have also found that patients with an HFpEF diagnosis according to current clinical criteria who also suffer from a compromised NO-cGMP-PKG signalling axis can be selected based on biomarkers such as their NADPH oxidase Type 5 (Nox5) plasma levels, nitration of tyrosine residues on their plasma proteins, or a cell-based assay. Moreover, the inventors have found that treating this signalling network at two or more nodes, especially sGC and NO synthase, achieves the first-in-class mechanism-based, causal and high precision therapy for HFpEF endotype which is defined by insufficient NO-cGMP-PKG signalling.

Without wishing to be bound by any particular theory, the present inventors believe that, while the Paulus & Tschöppe model is quite accurate in its basic assumption of an impaired NO-cGMP-PKG axis, the amount of reactive oxygen species (ROS) generated by the proinflammatory state induced by the metabolic syndrome or its components (obesity, diabetes, and hypertension) would in many cases not be sufficient to impair the NO-cGMP-PKG axis to the extent necessary for HFpEF development. Rather, this would only be the case if NADPH oxidase type 5 (Nox5), a strongly ROS-generating enzyme, is overexpressed or overactive in endothelial cells, as seen in an endotype of age-related systolic hypertension (PLOS Biol. 2020; 18 (11): e3000885). These initially mostly Nox5-generated ROS would uncouple cardiac NOS by depleting tetrahydrobiopterin, causing it to produce superoxide anion instead of NO; this is a unique feature of this class of enzymes that was suggested as a cause of diastolic dysfunction (Circulation. 2010; 121 (4): 519-28) but without reference to the crucial role of Nox5. At the same time, superoxide and other ROS will further deplete NO directly by converting it to peroxynitrite, which generates more ROS (see FIG. 1), setting up a vicious cycle that leads to HFpEF development. FIG. 8 displays the NOX5 plasma levels in HFpEF patients according to two HFpEF classification scores i.e., the H2FPEF and the HFAPEFF scores. In HFpEF patients determined by the HFAPEFF score, a subgroup or endotype of patients with high NOX5 levels (>=110 ng/ml) can be distinguished. This endotype corresponds with roughly 25% of all the HFpEF patients according to the HFAPEFF score. FIG. 9 displays the 3-NT plasma levels in HFpEF patients according to two HFpEF classification scores i.e., the H2FPEF and the HFAPEFF scores. 5 out of 7 patients that had high NOX5 levels also show high levels of 3-NT. Based on these findings and the above-mentioned hypothesis, the current invention resides, in one aspect, in the treatment of a specific subgroup of subjects suffering from or at risk of suffering from HFpEF, notably subjects that have an impaired NO-cGMP-PKG axis, and, in a further aspect, in the treatment of HFpEF, especially in said subgroup of patients having an impaired NO-cGMP-PKG axis, using combinations of active agents that target distinct nodes in the NO-cGMP-PKG axis. More in particular, in accordance with preferred embodiments of the present invention, patients preselected by clinical HFpEF scores are endotyped as having an impaired NO-cGMP-PKG axis. In accordance with the invention, HFpEF patients, especially those endotyped as having an impaired NO-cGMP-PKG axis, are preferably treated as follows: i) an sGC modulator is administered to reactivate and/or stimulate sGC so that normal amounts of cGMP can be synthesized from the NO that now becomes available again; ii) one or more agent(s) that reverse NOS uncoupling are administered so that NOS can resume NO production instead of producing additional ROS; iii) excess NOS substrate or a precursor is administered so that further uncoupling of the newly recoupled NOS is avoided.

Hence, a first aspect of the invention concerns a method for the prophylactic and/or therapeutic treatment of a subject suffering from or at risk of suffering from HFpEF, preferably a subject having an impaired NO-cGMP-PKG axis, said method comprising the administration to said subject of a pharmaceutical composition comprising an sGC (positive) modulator, typically in combination with a NOS recoupler and/or a NOS substrate (precursor).

A further aspect of the invention concerns a pharmaceutical composition comprising an sGC (positive) modulator, for use in a method for the prophylactic and/or therapeutic treatment of a subject suffering from or at risk of suffering from HFpEF, preferably a subject having an impaired NO-cGMP-PKG axis, said method comprising the administration to said subject of the pharmaceutical composition comprising the sGC (positive) modulator, typically in combination with a NOS recoupler and/or a NOS substrate (precursor).

Yet, a further aspect of the invention concerns the use of an sGC (positive) modulator, in the manufacture of a pharmaceutical composition for use in a method for the prophylactic and/or therapeutic treatment of a subject suffering from or at risk of suffering from HFpEF, preferably a subject having an impaired NO-cGMP-PKG axis, said method comprising the administration to said subject of the pharmaceutical composition comprising the sGC (positive) modulator, typically in combination with a NOS recoupler and/or a NOS substrate (precursor).

Other aspects of the invention concern diagnostic methods, for determining whether a subject suffers from the HFpEF endotype of the invention, pharmaceutical compositions, preferably in unit dosage form, comprising an sGC (positive) modulator, preferably in combination with a NOS recoupler and/or a NOS substrate (precursor); and kits comprising a package containing a plurality of one or more of such pharmaceutical unit dosage forms as well as a leaflet containing printed instructions to repeatedly self-administer said unit dosage forms in order to treat and/or prevent HFpEF, preferably in a subject having an impaired NO-cGMP-PKG axis.

Specific details and preferred embodiments of the afore-mentioned methods as well as of the compositions and pharmaceutical kits used therein will become evident to those skilled in the art on the basis of the following detailed description and the appended experimental part.

### DETAILED DESCRIPTION OF THE INVENTION

### Pharmaceutical Compositions

Pharmaceutical compositions in accordance with the present invention comprise, as a first active ingredient (‘API1’), a positive modulator, typically a stimulator or activator, of soluble guanylate cyclase (‘sGC’). As used herein, the terms “sGC modulator” or “sGC positive modulator” are used to refer to agents that increase the enzymatic activity of soluble guanylate cyclase to generate cGMP, independently of NO, either by acting on the holoenzyme (referred to in the art as “sGC stimulators” and/or ‘sGCs’) or on the apoenzyme which has lost its heme functional group (referred to in the art as “sGC reactivators”, “sGC activators” and/or “sGCa”). Suitable examples of compounds that (positively) modulate sGC include riociguat, vericiguat, ataciguat, neliciguat, etriciguat, lificiguat, IW-1701, IW-1973, IWP-051, IWP-121, IWP-427, IWP-953, BAY-60-2770, A-344905, A-350619, A-778935, BI-684067, BI-703704, BAY-41-2272, BAY-41-8543, BAY 60-4552, CF-1571, cinaciguat and HMR-1766.

In accordance with particularly preferred embodiments of the invention, the sGC (positive) modulator is riociguat or vericiguat or a pharmaceutically acceptable salt, hydrate or solvate thereof. Riociguat is the INN of the compound having the IUPAC name Methyl N-[4,6-Diamino-2-[1-[(2-fluorophenyl)methyl]-1H-pyrazolo[3,4-b]pyridin-3-yl]-5-pyrimidinyl]-N-methyl-carbaminate and structural formula (I) as depicted below. Vericiguat is the INN of the compound having the IUPAC name methyl N-[4,6-diamino-2-[5-fluoro-1-[(2-fluorophenyl)methyl]pyrazolo[3,4-b]pyridin-3-yl]pyrimidin-5-yl]carbamate and structural formula (II) as depicted below.

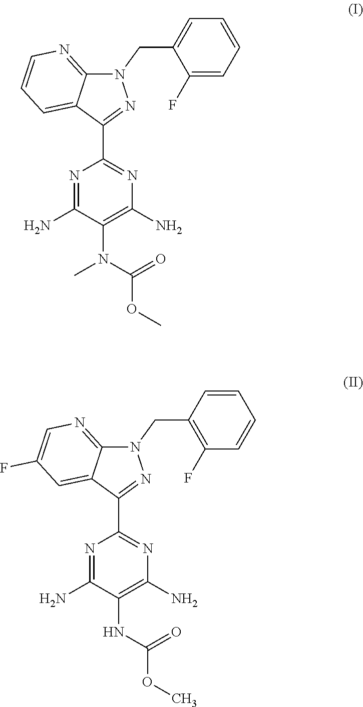 [SMILES: COC(=O)N(C)c1c(N)nc(-c2nn(Cc3ccccc3F)c3ncccc23)nc1N ; COC(=O)Nc1c(N)nc(-c2nn(Cc3ccccc3F)c3ncc(F)cc23)nc1N]

The term “pharmaceutically acceptable” as used herein has its conventional meaning and refers to compounds, materials, compositions and/or dosage forms, which are, according to sound medical judgment, considered suitable for contact with the tissues of mammals, especially humans, without causing excessive toxicity, irritation, allergic response and other complications, commensurate with a reasonable benefit/risk ratio.

As used herein, “a pharmaceutically acceptable salt” includes any salt that retains the activity of the active agent(s) and is acceptable for pharmaceutical use. The pharmaceutically acceptable salt of the disclosed compounds may be prepared by methods well known to those skilled in the art.

Furthermore, the compositions can comprise the active ingredients in the form of a solvate, comprising a pharmaceutically acceptable solvent, such as water (‘hydrate’), ethanol, and the like. In general, the solvated forms are considered equivalent to the unsolvated forms for the purposes of the present invention.

As used herein, the term “pharmaceutical composition” refers to a composition comprising the sGC (positive) modulator and, as the case may be, one or more additional, non-toxic ingredients, which composition is in a form suitable for administration to a (human) subject, through any route of administration, and which composition is physiologically tolerated upon such administration.

In a preferred embodiment, the composition comprises one or more carriers and/or excipients. As is known by those of average skill in the art, the appropriate choice of excipients is dependent on multiple factors, including the physicochemical properties of the API, the preferred pharmaceutical form, the preferred route of administration, the desired rate of release, etc. The compositions of the invention can be formulated for a variety of routes of administration, oral administration being particularly preferred. Pharmaceutical compositions adapted for oral administration may be presented as discrete units such as capsules or tablets, as powders or granules to be dissolved before use, as solutions, syrups or suspensions, or edible foams or whips; or as emulsions. Suitable excipients for tablets or hard gelatine capsules include lactose, maize starch or derivatives thereof, stearic acid or salts thereof. Suitable excipients for use with soft gelatine capsules include, for example, vegetable oils, waxes, fats, semi-solid or liquid polyols etc. For the preparation of solutions and syrups, excipients that may be used include, for example, water, polyols and sugars. It is within the purview of those of average skill in the art to conceive and develop suitable formulations, relying on the common general knowledge as reflected in text books such as Remington's Pharmaceutical Sciences (Meade Publishing Co., Easton, Pa., 20th Ed., 2000), the entire disclosure of which is herein incorporated by reference, and routine development efforts.

In accordance with the various aspects of the invention, the pharmaceutical composition is preferably provided in unit dosage form. The term “unit dosage form” refers to a physically discrete unit that is suitable for administration to a human subject, each unit containing a predetermined quantity of active material calculated to produce the desired therapeutic effect in association with any suitable pharmaceutical carrier(s) and/or excipient(s). Exemplary, non-limiting unit dosage forms include a tablet, caplet, capsule (e.g., a hard capsule or a soft capsule), lozenge, film, strip, gelcap as well as any metered volume of a solution, suspension, syrup or elixir or the like, which may be contained, for instance in a vial, syringe, applicator device, sachet, spray, micropump etc. In accordance with particularly preferred embodiments of the invention, the unit dosage form, is a unit dosage form that is suitable for oral administration. Most preferably, it is a solid unit dosage form, such as a tablet for oral ingestion.

In accordance with the various aspects of the invention, the pharmaceutical composition is provided in a unit dosage form comprising the sGC (positive) modulator in a dose of at least 0.01 mg, preferably at least 0.05 mg, at least 0.1 mg, at least 0.2 mg, at least 0.25 mg, at least 0.3 mg, at least 0.4 mg, or at least 0.5 mg. In accordance with the various aspects of the invention, the composition is typically provided in a unit dosage form comprising the sGC (positive) modulator in a dose of 100 mg or less, more preferably 75 mg or less, 50 mg or less, 40 mg or less, 30 mg or less, 20 mg or less, 15 mg or less, 12.5 mg or less, 12 mg or less, or 11 mg or less. In accordance with the various aspects of the invention, the composition is preferably provided in a unit dosage form comprising the sGC (positive) modulator in a dose within the range of 0.1-100 mg, 0.2-50 mg, 0.3-50 mg, 0.4-25 mg, 4 or 0.5-10 mg.

In accordance with the various aspects of the invention, the pharmaceutical composition is provided in a unit dosage form comprising vericiguat in a dose of at least 0.1 mg, preferably at least 0.25 mg, at least 0.5 mg, at least 1 mg, at least 1.25 mg, at least 1.5 mg, at least 2 mg, or at least 2.5 mg; or a salt, solvate or hydrate of vericiguat in the equipotent dose. In accordance with the various aspects of the invention, the composition is typically provided in a unit dosage form comprising vericiguat in a dose of 100 mg or less, more preferably 75 mg or less, 50 mg or less, 40 mg or less, 30 mg or less, 20 mg or less, 15 mg or less, 12.5 mg or less, 12 mg or less, or 11 mg; or a salt, solvate or hydrate of vericiguat in the equipotent dose. In accordance with the various aspects of the invention, the composition is preferably provided in a unit dosage form comprising vericiguat in a dose within the range of 0.5-100 mg, 1-50 mg, 1.5-50 mg, 2-25 mg, or 2.5-10 mg; or a salt, solvate or hydrate of vericiguat in the equipotent dose.

In accordance with the various aspects of the invention, the composition is provided in a unit dosage form comprising riociguat in a dose of at least 0.01 mg, preferably at least 0.05 mg, at least 0.1 mg, at least 0.2 mg, at least 0.25 mg, at least 0.3 mg, at least 0.4 mg, or at least 0.5 mg; or a salt, solvate or hydrate of riociguat in the equipotent dose. In accordance with the various aspects of the invention, the composition is typically provided in a unit dosage form comprising riociguat in a dose of 50 mg or less, more preferably 25 mg or less, 20 mg or less, 15 mg or less, 10 mg or less, 7.5 mg or less, 5 mg or less, 4 mg or less, 3 mg or less, or 2.5 mg; or a salt, solvate or hydrate of riociguat in the equipotent dose. In accordance with the various aspects of the invention, the composition is preferably provided in a unit dosage form comprising riociguat in a dose within the range of 0.1-50 mg, 0.2-25 mg, 0.3-10 mg, 0.4-5 mg, 4 or 0.5-2.5 mg; or a salt, solvate or hydrate of riociguat in the equipotent dose.

As used herein, the term “equipotent” means equally potent or equally capable of producing a pharmacologic effect of certain intensity. It is also common in the art to refer to amounts of a given compound ‘equivalent’ to a specified amount of a reference compound. For example, if the composition comprises a salt of the sGC modulator, the amount of said salt to be administered and/or to be incorporated into a unit dose form needs to be adjusted to take account of the molecular weight difference between the free base and salt form. In expressing dose amounts in the label and/or product information of authorized medicinal products comprising a salt form of an active compound that can also be used in free base form, it is customary practice to specify the dose of the free base to which the dose of the salt as used is equivalent. In this context, the term ‘equipotent’ is deemed synonymous to the term ‘equivalent’.

In accordance with the various aspects of the invention, the methods of treatment comprises the administration of a NOS recoupling agent, as explained/defined herein before. The term ‘NOS recoupling’ is used herein to refer to the phenomenon that Nitric oxide synthase enzyme (NOS) can become “uncoupled” to produce superoxide anion (O(2)(−)) instead of nitric oxide (NO) and that NOS recoupling can be effected by providing sufficient levels of tetrahydrobiopterin to replenish the depleted stores that caused uncoupling (Free Radic Biol Med. 2013; 65:234-43). In one embodiment of the invention, NOS recoupling is attained by the administration of folate. As will be explained herein elsewhere, the NOS recoupling agent may be administered as a separate formulation or it may be combined with the sGC (positive) modulator in a ‘fixed dose combination product’.

Hence, in preferred embodiments of the present invention, the pharmaceutical compositions as defined herein, comprise, as a further active ingredient (‘API2’), a NOS recoupling agent, preferably a NOS recoupling agent selected from the group consisting of folic acid and pharmaceutically acceptable salts thereof. The term “folate” may be used herein to refer to folate (the active form) and, more specifically, to its prodrug folic acid (pteroylmonoglutamic acid or pteroylglutamic acid) in all pharmaceutically acceptable forms.

In accordance with the various aspects of the invention, such pharmaceutical compositions are provided in a unit dosage form comprising the NOS recoupling agent, preferably folate, in a dose of at least 0.01 mg, preferably at least 0.05 mg, at least 0.1 mg, at least 0.2 mg, at least 0.25 mg, at least 0.3 mg, at least 0.4 mg, or at least 0.5 mg. In accordance with the various aspects of the invention, the composition is typically provided in a unit dosage form comprising the NOS recoupling agent, preferably folate, in a dose of 100 mg or less, more preferably 75 mg or less, 50 mg or less, 40 mg or less, 30 mg or less, 20 mg or less, 15 mg or less, 12.5 mg or less, 12 mg or less, or 11 mg or less. In accordance with the various aspects of the invention, the composition is preferably provided in a unit dosage form comprising the NOS recoupling agent, preferably folate, in a dose within the range of 0.1-100 mg, 0.2-50 mg, 0.3-50 mg, 0.4-25 mg, 4 or 0.5-10 mg.

In accordance with the various aspects of the invention, the methods or treatment comprises the administration of a NOS substrate or a precursor thereof, as explained/defined herein before. In one embodiment, the NOS substrate is L-arginine. In another embodiment, the NOS substrate is L-citrulline, which is a natural precursor of L-arginine and is more bioavailable than L-arginine because it avoids hepatic first-pass metabolism and has a longer residence time in the systemic circulation. In yet another embodiment, L-arginine and L-citrulline are administered together. In accordance with the invention, the L-citrulline, as well as the L-arginine may be administered any pharmaceutically and/or nutritionally acceptable form, especially in the form of a pharmaceutically and/or nutritionally acceptable salt, hydrate or solvate. As explained herein, the NOS substrate (precursor) may be administered as a separate formulation or it may be combined with the sGC (positive) modulator and/or the NOS recoupler, to provide a (binary or ternary) ‘fixed dose combination product’.

Hence, in embodiments of the present invention, the pharmaceutical composition as defined herein, comprises, as a further active ingredient (‘API3’), a NOS substrate or NOS substrate precursor, preferably a NOS substrate (precursor) selected from the group consisting of L-citrulline, L-arginine, and pharmaceutically acceptable salts, hydrates and solvates thereof, and combinations thereof.

In accordance with the various aspects of the invention, such pharmaceutical composition is provided in a unit dosage form comprising the NOS substrate (precursor), preferably L-citrulline or a salt thereof, in a dose of at least 0.1 mg, preferably at least 0.25 mg, at least 0.5 mg, at least 1 mg, at least 1.25 mg, at least 1.5 mg, at least 2 mg, or at least 2.5 mg. In accordance with the various aspects of the invention, the composition is typically provided in a unit dosage form comprising the NOS substrate, preferably L-citrulline or a salt thereof, in a dose of 100 mg or less, more preferably 75 mg or less, 50 mg or less, 40 mg or less, 30 mg or less, 20 mg or less, 15 mg or less, 14 mg or less, 13 mg or less, 12.5 mg or less or 12 mg or less. In accordance with the various aspects of the invention, the composition is preferably provided in a unit dosage form comprising the NOS substrate, preferably L-citrulline or a salt thereof, in a dose within the range of 0.1-50 mg, 0.5-25 mg, 1-20 mg, 2-15 mg, or 3-12 mg.

### Therapeutic Indications

As explained herein before, the invention provides methods for the curative and/or prophylactic treatment of a subject in need of such treatment, wherein the treatment comprises the administration of an sGC modulator, preferably in combination with a NOS recoupler and/or a NOS substrate (precursor). More in particular, the invention provides methods for the treatment and/or prevention of heart failure with preserved ejection fraction (HFpEF) in such subjects. The invention further provides methods for the treatment and/or prevention of one or more symptoms associated with HFpEF, in such subjects, by the administration of the sGC modulator, preferably in combination with the NOS recoupler and/or the NOS substrate (precursor). The invention further provides methods for the treatment and/or prevention of one or more pathologies associated with and/or caused by HFpEF, in such subjects, by the administration of the sGC modulator, preferably in combination with the NOS recoupler and/or the NOS substrate (precursor).

As used herein, the term “heart failure with preserved ejection fraction” and its abbreviation “HFpEF” mean a type of heart failure, diagnosed e.g. according to current clinical and blood chemistry consensus criteria, with an ejection fraction that is at least 50 percent of the nominal value. Non-limiting examples of HFpEF consensus criteria include those of the European Society of Cardiology HFA-PEFF algorithm (Eur Heart J. 2019; 40 (40): 3297-317); the H2FPEF score as published by Reddy et al. (Circulation. 2018; 138 (9): 861-70); or any of the other diagnostic algorithms discussed by Kaplon-Cieślicka et al. (Cardiol J. 2020; 27 (5): 449-68).

The terms “treat”, “treating” or “treatment”, when used in conjunction with a specific disease or symptom (for example: “method of treating disease . . . ”) refers to curing, alleviating or abrogating said disease and/or accompanying symptoms, diminishing extent of disease, stabilizing (i.e. not worsening) the state of disease, delaying or slowing of disease progression, ameliorating the disease state, prolonging survival (as compared to expected survival without treatment), etc. The terms “prevent”, “preventing” or “prevention”, as used herein, refer to reducing the risk for a subject to acquire a disease and/or accompanying symptoms, delaying the moment a subject acquires disease, etc. The terms “treat”, “treating” or “treatment”, when used in relation to a patient or subject (for example: “method of treating a subject”), typically refers to the act of administering a therapeutic compound to said patient or subject for whatever therapeutic and/or prophylactic purpose.

### Subjects to be Treated

As explained herein before, the methods of the invention are directed at the treatment and/or prevention of a subject suffering from or at risk of suffering from HFpEF.

The term “a subject” refers to a living organism, typically a mammal, in particular a human subject. In one embodiment of the invention, the subject is human male. In another embodiment of the invention, the subject is human female.

In further preferred embodiments of the invention, the subject is at increased risk based on age, such as a subject being over 35 years of age, over 40 years of age, over 45 years of age, over 50 years of age, over 55 years of age, over 60 years of age, over 65 years of age or over 70 years of age; typically in combination with one or more other risk factors as defined herein.

In particularly preferred embodiments of the invention, the subject is a subject that is suffering from and/or has been diagnosed with HFpEF.

In certain embodiments of the invention, the subject is a subject with a H₂FPEF score of 3 or higher, 4 or higher, 5 or higher or 6 or higher, wherein H₂FPEF score refers to the subject's score in the scoring system developed and published by Reddy et al. (Circulation. 2018; 138 (9): 861-70).

In certain preferred embodiments of the invention, the subject is a subject with a HFAPEFF score of 4 or higher, preferably 5 or higher, wherein HFAPEFF score refers to the subject's score in the scoring system developed and published by the Heart Failure Association (HFA) of the European Society of Cardiology (ESC) (Eur. Heart J. 2019; 40:3297-3317).

In further preferred embodiments of the invention, the subject is a subject that is considered to be at risk, typically at above-average risk, of attracting or developing HFpEF. In preferred embodiments of the invention, the subject is a subject suffering from one or more conditions known to bear a causal and/or epidemiological correlation with the occurrence of HFpEF, such as anemia, atrial fibrillation, chronic kidney disease, chronic obstructive pulmonary disease (COPD), coronary artery disease, diabetes, hypertension (high blood pressure), Inflammatory or autoimmune diseases, obesity, sleep apnea, etc. In further preferred embodiments of the invention, the subject is a subject that is genetically predisposed to develop HFpEF. In further preferred embodiments of the invention, the subject is a subject prone to develop HFpEF as a consequence of life-style/habitual factors.

In some embodiments of the invention, the subject to be treated is normotensive. As used herein, the term ‘normotensive’ refers to a blood pressure within the range considered normal, according to recommendations by the American College of Cardiology (ACC)/American Heart Association (AHA) or according to the recommendations by the European Society of Cardiology (ESC)/European Society of Hypertension (ESH). In accordance with the invention, normotensive typically refers to subjects having a systolic pressure of 129 mm Hg or lower, preferably 120 mm Hg or lower and/or a diastolic pressure of 84 mm Hg or lower, preferably 80 mm Hg or lower.

In some embodiments of the invention, the subject to be treated is hypertensive. As used herein, the term ‘hypertensive’ refers to a blood pressure that is elevated according to recommendations by the American College of Cardiology (ACC)/American Heart Association (AHA) or according to the recommendations by the European Society of Cardiology (ESC)/European Society of Hypertension (ESH). In accordance with embodiments of the invention, ‘hypertensive’ typically refers to subjects having a systolic pressure of 130 mm Hg or above, such as 139 mm Hg or above or 140 mm Hg or above, e.g. within the range of 130-180 mm Hg or within the range of 130-179 mm Hg, and/or by a diastolic pressure of 80 mm Hg or above, 85 mm Hg or above, or 90 mm Hg or above, such as within the range of 80-110 mm Hg, within the range of 85-110 mg Hg, within the range of 89-110 mm Hg or within the range of 90-110 mm Hg.

In accordance with embodiments of the invention, ‘hypertensive’ refers to subjects suffering from elevated blood pressure, hypertension stage 1, hypertension stage 2, according to recommendations by ACC/AHA, as defined in the table here below. In other embodiments of the invention, ‘hypertensive’ refers to subjects having a blood pressure graded as ‘normal’ (above optimal), ‘high normal’, grade 1 hypertension, grade 2 hypertension or grade 3 hypertension, preferably ‘high normal’, grade 1 hypertension, grade 2 hypertension or grade 3 hypertension, according to recommendations by ESC/ESH, as defined in the table here below. In accordance with embodiments of the invention, ‘normotensive’ refers to subjects having a blood pressure graded as ‘normal’ according to recommendations by ACC/AHA, as defined in the table here below. In other embodiments of the invention, ‘normotensive’ refers to subjects having a blood pressure graded as ‘optimal’ or ‘normal’, preferably ‘optimal’, according to recommendations by ESC/ESH, as defined in the table here below.

| | ACC/AHA | ESC/ESH |
|---|---|---|
| Definition of | ≥130/80 | ≥140/90 |
| hypertension | | |
| (mm Hg) | | |
| Normal blood | Normal: <120/80 | Optimal: <120/80 |
| pressure range | Elevated: 120-129/<80 | Normal: 120-129/80-84 |
| (mm Hg) | | High normal: 130-139/85-89 |
| Hypertension | Stage 1: 130-139/80-89 | Grade 1: 140-159/90-99 |
| stages | Stage 2: ≥140/90 | Grade 2: 160-179/100-109 |
| (mm Hg) | | Grade 3: ≥180/110 |

Furthermore, as will be apparent from the foregoing, the subject to be treated in accordance with the invention, typically has an impaired NO-cGMP-PKG signalling axis. As will be understood by those skilled in the art, based on the present teachings, impairment in the NO-cGMP-PKG axis functioning can be established relying on biomarker based criteria, although the invention is not particularly limited in this regard. In an exemplary embodiment, impairment in the NO-cGMP-PKG axis functioning is established by obtaining a blood sample from a subject; processing the sample to obtain a plasma/serum/exosome sample and/or a cell/exosome sample; and determining one or more of the following biomarker based criteria:
A) expression level of NADPH oxidase type 5 (Nox5) in the plasma/serum/exosome sample;
B) level of nitrotyrosine in the plasma/serum/exosome sample; In a non-limiting fashion, the methodology to make determinations A) and/or B) can be as follows:
   Whole blood is collected in a tube with or without anticoagulant by venipuncture. Without delay, blood is centrifuged at 800×g for 10 minutes.
   Plasma/serum/exosomes may be collected and stored at −80° C. until biomarker measurement.
   Next, plasma/serum/exosomes samples are diluted accordingly and biomarker levels measured using standardized and commercially available antibodies or ELISA kits.

In accordance with a preferred embodiment of the invention, the subject to be treated has an increased NOX5 level, compared to average NOX5 levels in healthy subjects, such as a NOX5 plasma level of at least 90 ng/ml, at least 95 ng/ml, at least 100 ng/mL, at least 102.5 ng/mL, at least 105 ng/ml, at least 107.5 ng/mL, at least 110 ng/mL, at least 112.5 ng/ml, at least 115 ng/ml, at least 117.5 ng/mL or at least 120 ng/ml. In accordance with the present invention, NOX5 concentrations can be determined by ELISA.

In accordance with a preferred embodiment of the invention, the subject to be treated has a nitrotyrosine level of at least 500 nM, more preferably at least 600 nM, at least 800 nM, at least 1000 nM, or at least 1200 nM. In accordance with the present invention, nitrotyrosine concentrations can be determined by ELISA.

In a further exemplary embodiment, impairment in the NO-cGMP-PKG axis functioning is established by obtaining a blood sample from a subject; processing the sample to obtain a cell and/or exosome sample and quantitatively determining the extent to which cGMP synthesis and/or downstream signalling phosphorylation responses can be stimulated in the cell and/or exosome sample. In a non-limiting fashion, the methodology to make this determination may be as follows:
After whole blood collection by venipuncture, blood is centrifuged and different fractions containing cells and/or exosomes are isolated, e.g. buffy coat).
Those fractions may be cryopreserved accordingly, i.e. with addition of cryopreserving reagents and storage at −80° C.
After specific thawing procedures, these cell and/or exosome samples are exposed to cGMP modulating drug compounds and cGMP signaling is evaluated by measuring the cGMP production and/or the phosphorylation response.

In certain embodiments of the invention, the methodology involves determining the induction of cGMP synthesis in response to an NO donor and/or induction of PKG-dependent protein phosphorylation in response to an NO donor. As is known by those skilled in the art, cGMP synthesis can be (quantitatively) determined by ELISA. As is known by those skilled in the art, PKG-dependent protein phosphorylation can be (quantitatively) determined by Western blot.

In accordance with a preferred embodiment of the invention, the subject's impaired response to stimulation of cGMP synthesis, is reflected by a level of cGMP production in the cells and/or exosomes containing sample in response to an NO donor that is below a predetermined reference value, e.g. less than 90%, preferably less than 80%, less than 70%, less than 60%, less than 50% of the predetermined reference value.

As used herein, in the context of establishing alterations in the extent to which cGMP synthesis can be stimulated, the term “pre-determined reference value” refers to a threshold value or a cut-off value that distinguishes the normal, non-pathological state from a pathological state, where values above the threshold are indicative of the normal, non-pathological endotype and values below the threshold are indicative of the pathological endotype (or vice versa). Typically, such a “threshold value” or “cut-off value” can be determined experimentally, empirically, or theoretically. In embodiments of the invention wherein the pathological state is characterized by a decrease in the response to a certain stimulus (compared to a healthy, non-pathologic state), the predetermined reference value may be the 10^{th} percentile cut-off point as established in a normal, non-pathological reference population of sufficient size, more preferably the 5^{th} percentile cut-off point, the 4^{th} percentile cut-off point, the 3^{th} percentile cut-off point, the 2^{nd} percentile cut-off point or the 1^{st} percentile cut-off point as established in a normal, non-pathological reference population of sufficient size using the same test procedure. In embodiments of the invention wherein the pathological state is characterized by an increase in the response to a certain stimulus (compared to a healthy, non-pathologic state), the predetermined reference value may be the 90^{th} percentile cut-off point as established in a normal, non-pathological reference population of sufficient size, more preferably the 95^{th} percentile cut-off point, the 96^{th} percentile cut-off point, the 97^{th} percentile cut-off point, the 98^{th} percentile cut-off point or the 99^{th} percentile cut-off point as established in a normal, non-pathological reference population of sufficient size using the same test procedure.

In accordance with another preferred embodiment of the invention, the subject's impaired response to stimulation of cGMP synthesis is reflected by a level of PKG-dependent protein phosphorylation in the cells and/or exosomes containing sample in response to an NO donor that is below a pre-determined reference value, e.g. less than 90%, less than 80%, less than 70%, less than 60%, or less than 50% of the pre-determined reference value

In certain embodiments of the invention, the methodology involves determining the induction of cGMP synthesis in response to an sGC stimulator and/or induction of PKG-dependent protein phosphorylation in response to an sGC stimulator. In accordance with a preferred embodiment of the invention, the subject's impaired response to stimulation of cGMP synthesis, is reflected by a level of cGMP production in the cells and/or exosomes containing sample in response to an sGC stimulator that is below a pre-determined reference value, e.g. less than 90%, less than 80%, less than 70%, less than 60%, or less than 50% of the pre-determined reference value. In accordance with another preferred embodiment of the invention, the subject's impaired response to stimulation of cGMP synthesis is reflected by a level of PKG-dependent protein phosphorylation in the cells and/or exosomes containing sample in response to an sGC stimulator that is below a pre-determined reference value, e.g. less than 90%, less than 80%, less than 70%, less than 60%, or less than 50% of the pre-determined reference value.

In certain embodiments of the invention, the methodology involves determining the induction of cGMP synthesis in response to an sGC activator and/or induction of PKG-dependent protein phosphorylation in response to an sGC activator. In accordance with a preferred embodiment of the invention, the subject's impaired response to stimulation of cGMP synthesis, is reflected by a level of cGMP production in the cells and/or exosomes containing sample in response to an sGC activator that is above a pre-determined reference value, e.g. more than 10%, more than 20%, more than 30%, more than 40%, or more than 50% of the pre-determined reference value. In accordance with another preferred embodiment of the invention, the subject's impaired response to stimulation of cGMP synthesis is reflected by a level of PKG-dependent protein phosphorylation in the cells and/or exosomes containing sample in response to an sGC activator that is above a pre-determined reference value, e.g. more than 10%, more than 20%, more than 30%, more than 40%, or more than 50% of the pre-determined reference value.

In accordance with a preferred embodiment of the invention, the subject's impaired response to stimulation of cGMP synthesis, is reflected by a ratio between i) the level of cGMP production in the cells and/or exosomes containing sample in response to an sGC stimulator and ii) the level of cGMP production in the cells and/or exosomes containing sample in response to an sGC activator, that is below a pre-determined reference value, e.g. less than 90%, less than 80%, less than 70%, less than 60%, or less than 50% of the pre-determined reference value. In accordance with another preferred embodiment of the invention, the subject's impaired response to stimulation of cGMP synthesis is reflected by a ratio between i) the level of PKG-dependent protein phosphorylation in the cells and/or exosomes containing sample in response to an sGC stimulator and ii) the level of PKG-dependent protein phosphorylation in the cells and/or exosomes containing sample in response to an sGC activator, that is below a pre-determined reference value, e.g. less than 90%, less than 80%, less than 70%, less than 60%, or less than 50% of the pre-determined reference value. In accordance with preferred embodiments of the invention, the ratio is determined in fresh sample (cell preparation) and the pre-determined reference value, as determined in samples from healthy/non-pathological subjects, is about 8, about 8.25, about 8.5, about 8.75, about 9, about 9.25, about 9.75 or about 10. In accordance with other preferred embodiments of the invention, the ratio is determined in cryopreserved and thawed sample and the pre-determined reference value, as determined in samples from healthy/non-pathological subjects, is about 5.75, about 6, about 6.25, about 6.5, about 6.75, about 7, about 7.25 or about 7.5.

In accordance with another preferred embodiment of the invention, the subject's impaired NO-cGMP-PKG axis functioning is reflected by the subject's sGC oxidation status. The oxidation status can be assessed by comparing cGMP-dependent protein phosphorylation ex vivo in response to sGC stimulators and sGC activators. In a particularly preferred embodiment, this ratio, referred to herein as the sGCa/sGCs ratio, is determined in a P-WBC (platelet-enriched white-blood-cell) sample obtained from the subject, by measuring the pVASP response (pVASP/VASP) to 1 μM runcaciguat (an sGCa), in the presence of 500 μM IBMX (3-isobutyl-1-methylxanthine), independently measuring the pVASP response (pVASP/VASP) to 100 μM riociguat (an sGCs), in the presence of 500 μM IBMX, and dividing these respective responses to obtain the sGCa/sGCs ratio. As will be understood by those skilled in the art, based on the present teachings, in this assay, cGMP is produced in response to the stimulus and activates PKG, which phosphorylates VASP. IBMX inhibits PDEs, which are responsible for cGMP degradation, thereby maintaining the produced cGMP levels. The set-up is described in more detail in the examples. In accordance with preferred embodiments of the invention, it entails the following steps:
(a) collecting P-WBC by centrifugation of whole blood at 800 g for 10 minutes, after which P-WBC is isolated and cryopreserved with 6% DMSO at −80° C.;
(b) thawing of P-WBC for 5 minutes at 37° C. and diluted 1:100 with JNL-buffer;
(c) pre-treating cells with IBMX (500 μM, 10 minutes);
(d) treating cells with runcaciguat (1 μM) or riociguat (100 μM) at 37° C. for 15 minutes;
(e) centrifuging samples for 10 minutes at 750 g;
(f) lysing cell pellets with ROTI®Load Laemmli supplemented with phosphatase and protease inhibitors;
(g) boiling lysates boiled for 5 minutes at 95° C.;
(h) separating cell lysates by SDS-PAGE;
(i) analysis by Western Blot using as primary antibodies anti-phosphoVASP Ser 239 (Nanotools) and total VASP (Enzo), wherein ECL detection is performed for signal visualisation and densitometric analysis is done reporting the response to the stimulus as relative intensity (a.u.) which reflects the phosphorylation of VASP.

In a non-limiting fashion, the methodology to obtain the P-WBC sample to be used for determining the sGCa/sGCs ratio can be as follows:
Whole blood is collected with or without anticoagulant, e.g. by venipuncture, following which blood is (immediately) centrifuged at speeds of 200-800xg, preferably 400-800xg, more preferably 600-800 xg and most preferably at about 800xg, for 5-20 minutes, more preferably 7.5-15 minutes, e.g. around 10 minutes;
Plasma is collected and P-WBC is isolated;
P-WBC are cryo-preserved by storing them at a temperature of between −60 to −90° C., preferably-70 to −85° C., most preferably at around −80° C., using a cryoprotectant, preferably DMSO, e.g. 6% DMSO; and
Cryopreserved P-WBC are thawed at a temperature of about 37° C., for a period of, e.g., about 5 minutes prior to determining the sGCa/sGCs ratio using the method as defined here above.

As illustrated in the examples below, a subgroup of patients with sGCa/sGCs values higher than 1.05 can be identified, in which the apo-sGC form of the enzyme is more prominent. Hence, in accordance with a preferred embodiment of the invention, the subject to be treated is characterized by a sGCa/sGCs ratio, in the assay as defined here above, that is above a certain predetermined threshold, such as a ratio of at least 1.05, at least 1.06, at least 1.07, at least 1.08, at least 1.09, at least 1.10, at least 1.11, at least 1.12, at least 1.13, at least 1.14 or at least 1.15. In particularly preferred embodiments of the invention, the subject to be treated meets a combination of the biomarker based criteria defined herein before.
NOX5 plasma/serum/exosome concentration as defined herein before; in combination with
decreased cGMP production and/or the phosphorylation in response to a stimulus as defined here above.

In particularly preferred embodiments of the invention, the subject to be treated meets the following criteria:
NOX5 plasma/serum/exosome concentration as defined herein before; in combination with
reduced cGMP synthesis and/or reduced PKG-dependent protein phosphorylation in response to an NO donor, as defined here above.

In particularly preferred embodiments of the invention, the subject to be treated meets the following criteria:
NOX5 plasma/serum/exosome concentration as defined herein before, in combination with
reduced cGMP synthesis and/or reduced PKG-dependent protein phosphorylation in response to an sGC stimulator, as defined here above.

In particularly preferred embodiments of the invention, the subject to be treated meets the following criteria:
NOX5 plasma/serum/exosome concentration as defined herein before, in combination with
increased cGMP synthesis and/or increased PKG-dependent protein phosphorylation in response to an sGC activator, as defined here above.

In particularly preferred embodiments of the invention, the subject to be treated meets the following criteria:
NOX5 plasma/serum/exosome concentration as defined herein before, in combination with
reduced ratio between i) the level of cGMP production in response to an sGC stimulator divided and ii) the level of cGMP production in response to an sGC activator and/or reduced pvas between i) the level of PKG-dependent protein phosphorylation in response to an sGC stimulator and ii) the level of PKG-dependent protein phosphorylation in response to an sGC activator, as defined here above.

In particularly preferred embodiments of the invention, the subject to be treated meets one or both of the following biomarker based criteria defined herein before:
a NOX5 plasma/serum/exosome concentration of at least 105 ng/ml; and/or
an sGCa/sGCs ratio, as can be determined using the assay as defined here above, of at least 1.05.

### Treatment Methods

The various aspects of the present invention as defined herein, all relate to methods of treatment involving the administration, typically the repeated administration, of the sGC (positive) modulator, typically in combination with the NO recoupler and/or NO substrate (precursor), as defined herein before, in a therapeutically effective doses.

As used herein, the terms “therapeutically acceptable amount” or “therapeutically effective dose” interchangeably refer to an amount of an agent or combination of agents that is sufficient to effect a reduction in the clinical symptoms of HFpEF or in laboratory parameters that are surrogate measures of HFpEF or the risk of developing HFpEF. In some embodiments, a “therapeutically acceptable amount” does not induce or cause undesirable side effects. Said amount can be determined by first administering a low dose and then incrementally increasing that dose until the desired result is achieved. In preferred embodiments of the invention, said methods involve the repeated administration of one or more pharmaceutical compositions or unit dosage forms as defined herein elsewhere, at a strength and frequency sufficient to effect a reduction in the clinical symptoms of HFpEF or in laboratory parameters that are surrogate measures of HFpEF or the risk of developing HFpEF.

Drugs according to the invention can be administered orally or parenterally; the oral route is particularly preferred.

Hence, in particularly preferred embodiments of the invention, the method comprises the administration, preferably the oral administration of the sGC modulator in a daily dose of at least 0.01 mg, preferably at least 0.05 mg, at least 0.1 mg, at least 0.2 mg, at least 0.25 mg, at least 0.3 mg, at least 0.4 mg, or at least 0.5 mg. In accordance with the various aspects of the invention, the method comprises the administration, preferably the oral administration, of the sGC modulator in a daily dose of 100 mg or less, preferably 75 mg or less, 50 mg or less, 40 mg or less, 30 mg or less, 20 mg or less, 15 mg or less, 12.5 mg or less, 12 mg or less, or 11 mg or less. In accordance with the various aspects of the invention, the method comprises the administration of the sGC modulator in a daily dose within the range of 0.1-100 mg, 0.2-50 mg, 0.3-50 mg, 0.4-25 mg, 4 or 0.5-10 mg.

In accordance with the various aspects of the invention, the method comprises the administration, preferably the oral administration, of vericiguat in a daily dose of at least 0.1 mg, preferably at least 0.25 mg, at least 0.5 mg, at least 1 mg, at least 1.25 mg, at least 1.5 mg, at least 2 mg, or at least 2.5 mg; or a salt, solvate or hydrate of vericiguat in the equipotent dose. In accordance with the various aspects of the invention, the method comprises the administration, preferably the oral administration, of vericiguat in a daily dose of 100 mg or less, more preferably 75 mg or less, 50 mg or less, 40 mg or less, 30 mg or less, 20 mg or less, 15 mg or less, 12.5 mg or less, 12 mg or less, or 11 mg; or a salt, solvate or hydrate of vericiguat in the equipotent dose. In accordance with the various aspects of the invention, the method comprises the administration, preferably the oral administration, of vericiguat in a daily dose within the range of 0.5-100 mg, 1-50 mg, 1.5-50 mg, 2-25 mg, or 2.5-10 mg; or a salt, solvate or hydrate of vericiguat in the equipotent dose.

In accordance with the various aspects of the invention, the method comprises the administration, preferably the oral administration, of riociguat in a daily dose of at least 0.01 mg, preferably at least 0.05 mg, at least 0.1 mg, at least 0.2 mg, at least 0.25 mg, at least 0.3 mg, at least 0.4 mg, or at least 0.5 mg; or a salt, solvate or hydrate of riociguat in the equipotent dose. In accordance with the various aspects of the invention, the method comprises the administration, preferably the oral administration, of riociguat in a daily dose of 50 mg or less, more preferably 25 mg or less, 20 mg or less, 15 mg or less, 10 mg or less, 7.5 mg or less, 5 mg or less, 4 mg or less, 3 mg or less, or 2.5 mg; or a salt, solvate or hydrate of riociguat in the equipotent dose. In accordance with the various aspects of the invention, the method comprises the administration, preferably the oral administration, of riociguat in a daily dose within the range of 0.1-50 mg, 0.2-25 mg, 0.3-10 mg, 0.4-5 mg, 4 or 0.5-2.5 mg; or a salt, solvate or hydrate of riociguat in the equipotent dose.

In accordance with the various aspects of the invention, the method comprises the administration, preferably the oral administration, of the NOS recoupling agent as defined herein, preferably folate, in a daily dose of at least 0.01 mg, preferably at least 0.05 mg, at least 0.1 mg, at least 0.2 mg, at least 0.25 mg, at least 0.3 mg, at least 0.4 mg, or at least 0.5 mg. In accordance with the various aspects of the invention, the method comprises the administration, preferably the oral administration, of the NOS recoupling agent, in a daily dose of 100 mg or less, more preferably 75 mg or less, 50 mg or less, 40 mg or less, 30 mg or less, 20 mg or less, 15 mg or less, 12.5 mg or less, 12 mg or less, or 11 mg or less. In accordance with the various aspects of the invention, the method comprises the administration, preferably the oral administration, of the NOS recoupling agent, in a daily dose within the range of 0.1-100 mg, 0.2-50 mg, 0.3-50 mg, 0.4-25 mg, 4 or 0.5-10 mg.

In accordance with the various aspects of the invention, the method comprises the administration, preferably the oral administration, of the NOS substrate (precursor) as defined herein, preferably L-citrulline and/or a salt thereof, in a daily dose of at least 0.1 mg, preferably at least 0.25 mg, at least 0.5 mg, at least 1 mg, at least 1.25 mg, at least 1.5 mg, at least 2 mg, or at least 2.5 mg. In accordance with the various aspects of the invention, the method comprises the administration, preferably the oral administration, of the NOS substrate, preferably L-citrulline and/or a salt thereof, in a daily dose of 100 mg or less, more preferably 75 mg or less, 50 mg or less, 40 mg or less, 30 mg or less, 20 mg or less, 15 mg or less, 14 mg or less, 13 mg or less, 12.5 mg or less or 12 mg or less. In accordance with the various aspects of the invention, the method comprises the administration, preferably the oral administration, of the NOS substrate, preferably L-citrulline and/or a salt thereof, in a daily dose within the range of 0.1-50 mg, 0.5-25 mg, 1-20 mg, 2-15 mg, or 3-12 mg.

As will be understood by those skilled in the art, based on the present teachings, it is not particularly critical whether the sGC modulator, the NOS recoupling agent and/or the NOS substrate (precursor) are administered conjunctly or sequentially and it is neither particularly critical, in the case of sequential administration, whether the respective agents are administered shortly/directly one after another or whether they are administered at different points in time, during the day. It will further be apparent to the skilled person, on the basis of the present teachings, that the daily doses of the respective active agents indicated herein may be contained in a single unit dosage form, or may be divided over a plurality of unit dosage forms, to be administered within certain interval throughout the day.

In certain embodiments of the invention, the method comprises the administration of the sGC modulator once or twice daily, at a total daily dose within the ranges defined here above, most preferably once daily. In further embodiments of the invention, the method comprises the administration of the NOS recoupling agent once or twice daily, at a total daily dose within the ranges defined here above, most preferably once daily. In further embodiments of the invention, the method comprises the administration of the NOS substrate (precursor) once, twice or three times a day, at a total daily dose within the ranges defined here above, most preferably once or twice a day.

It will be understood that, with a view to convenience for and/or compliance by the patient, it is preferred that the therapeutic agents are administered conjunctly or very shortly after another. For this purpose the use of the fixed dose combination products comprising at least two or all three of the active agents as defined herein elsewhere, is particularly preferred.

In one particularly preferred embodiment of the invention, the treatment comprises the administration of a single unit dosage form comprising the sGC modulator, the NOS recoupler and the NOS substrate (precursor), once, twice, three times or four times a day, at the total daily doses of the respective active agents as defined herein before, preferably one, twice or three times a day, more preferably once or twice a day, most preferably once a day.

In another particularly preferred embodiments of the invention, the treatment comprises the administration of a single unit dosage form comprising the sGC modulator and the NOS recoupler, once or twice a day, at the total daily doses of the respective active agents as defined herein before, preferably once a day; and the administration of a single unit dosage form comprising the NOS substrate (precursor), once, twice or three times a day, at the total daily doses as defined herein before, preferably twice a day.

Embodiments wherein unit dosage forms are used comprising higher amounts of the active agents than the daily dose indicated herein are also contemplated. This may e.g. involve the use of extended release dosage forms that remain in the body and keep releasing the active ingredient for a sufficiently period of time.

In accordance with the invention, the treatment comprises the administration of the sGC modulator, preferably in combination with a NOS recoupler and/or a NOS substrate (precursor), in accordance with the above-defined regimens, during a period of at least one month, at least three months, at least four months, at least six months, at least nine months, at least one year, at least two year, at least three year, at least 5 year, at least 10 year, at least 20 year, at least 30 year. There is no particular upper limit; treatment may be continued for as long as it is deemed beneficial to the subject's overall health and well-being (as determined by appropriately qualified healthcare professional), e.g. for the rest of the subject's life.

As will be apparent from the present teachings, the present methods are particularly suitable for the treatment of subjects suffering from HFpEF that have an impaired NO-cGMP-PKG axis. Hence, in certain embodiments of the invention, methods as defined herein are provided comprising a preceding or initial step of determining NO-cGMP-PKG axis functioning and/or of diagnosing impairment of NO-CGMP-PKG axis functioning in a subject, such as in a subject suffering from or at risk of suffering from HFpEF. In embodiments of the invention, said step of determining NO-cGMP-PKG axis functioning and/or diagnosing impairment of NO-cGMP-PKG axis functioning, comprises obtaining a blood sample from said subject; processing the blood sample to obtain a plasma/serum/exosome sample and/or sample containing cells and/or exosomes (‘cells/exosomes containing sample’); and determining one or more of the following biomarker based criteria:
A) expression level of NADPH oxidase type 5 (Nox5) in the plasma/serum/exosome sample;
B) nitrotyrosine level in the plasma/serum/exosome sample;
C) extent to which cGMP synthesis can be stimulated in the cells and or exosomes containing sample.

In a non-limiting fashion, the methodology to make determinations A) and B) can be as follows:
Whole blood is collected in a tube with or without anticoagulant by venipuncture. Without delay, blood is centrifuged at 800×g for 10 minutes.
Plasma/serum/exosomes are then collected and stored at −80° C. until biomarker measurement.
Next, plasma/serum/exosome samples are diluted accordingly and biomarker levels measured using standardised and commercially available antibodies or ELISA kits.

In a non-limiting fashion, the methodology to make determination C), i.e. an altered response to stimulation of cGMP synthesis in the cells and/or exosomes containing sample, can be as follows:
After whole blood collection by venipuncture, blood is centrifuged and different fractions containing cells and/or exosomes are isolated, e.g. buffy coat. Those fractions may be cryopreserved accordingly, i.e. with addition of cryopreserving reagents and storage at −80° C. After specific thawing procedures, these cell and/or exosome fractions are exposed to cGMP modulating drugs and cGMP signalling is evaluated by measuring the cGMP production and/or the phosphorylation response.

In particularly preferred embodiments of the invention, the method comprises the step of determining:
the subject's NOX5 plasma/serum/exosome concentration; and/or
the subject's nitrotyrosine plasma/serum/exosome level; and/or
the response to stimulation of cGMP synthesis in the subject's cells and/or exosomes;
   followed by the step of comparing the values with one or more pre-determined reference values, as defined herein elsewhere.

As will be understood by those skilled in the art, based on the present teachings, a determination that the subject suffering from HFpEF and/or at risk of suffering from HFpEF has impaired NO-cGMP-PKG axis functioning is typically followed by the step of treating the subject by the continuous/repeated administration of the sGC modulator, preferably in combination with a NOS recoupler and/or a NOS substrate (precursor), in accordance with the present teachings.

### Diagnostic Methods

In a further aspect of the invention, methods of diagnosing the HFpEF endotype of the present invention per se are provided. In preferred embodiments of the invention, such methods comprise the steps of i) obtaining a blood or plasma sample from a patient suffering from HFpEF; ii) analysing a biomarker characteristic of NO-cGMP-PKG axis functioning; and iii) determining that the patient suffers from HFpEF due to impaired NO-cGMP-PKG axis functioning in case the biomarker is above or below a pre-determined threshold level.

In one preferred embodiment of the invention, the biomarker is NOX5 and the method involved the following steps:
whole blood is collected with or without anticoagulant, e.g. by venipuncture, following which blood is (immediately) centrifuged;
plasma is collected and optionally stored at −80° C. until biomarker measurement;
the plasma sample NOX5 level is measured using a standardized and commercially available ELISA kit; and
a determination is made that the patient suffers from HFpEF due to impaired NO-cGMP-PKG axis functioning in case the NOX5 plasma level is at least 90 ng/ml, at least 95 ng/ml, at least 100 ng/ml, at least 102.5 ng/ml, at least 105 ng/mL, at least 107.5 ng/mL, at least 110 ng/mL, at least 112.5 ng/ml, at least 115 ng/ml, at least 117.5 ng/ml or at least 120 ng/ml.

In one preferred embodiment of the invention, the biomarker is damaged sGC and the method involves the following steps:
Whole blood is collected with or without anticoagulant, e.g. by venipuncture, following which blood is (immediately) centrifuged at speeds of 200-800xg, preferably 400-800xg, more preferably 600-800 xg and most preferably at about 800xg, for 5-20 minutes, more preferably 7.5-15 minutes, e.g. around 10 minutes;
Plasma is collected and P-WBC is isolated;
P-WBC are cryo-preserved by storing them at a temperature of between-60 to −90° C., preferably-70 to −85° C., most preferably at around −80° C., using a cryoprotectant, preferably DMSO, e.g. 6% DMSO; and
Cryopreserved P-WBC are thawed at a temperature of about 37° C., for a period of, e.g., about 5 minutes
The P-WBC sample is used to determine the pVASP response (pVASP/VASP) to 1 μM runcaciguat (an sGCa), in the presence of 500 μM IBMX (3-isobutyl-1-methylxanthine) as well as the pVASP response (pVASP/VASP) to 100 μM riociguat (an sGCs), in the presence of 500 M IBMX, typically using the methodology as described herein before;
a determination is made that the patient suffers from HFpEF due to impaired NO-cGMP-PKG axis functioning in case the ratio of the responses to the respective stimuli, referred to herein as the sGCa/sGCs ratio, is at least 1.05, at least 1.06, at least 1.07, at least 1.08, at least 1.09, at least 1.10, at least 1.11, at least 1.12, at least 1.13, at least 1.14 or at least 1.15.

An aspect of the invention concerns the method of preparing a P-WBC sample, which is suitable for determining the sGCa/sGCs ratio, per se. Hence a method of preparing a P-WBC sample is provided comprising the steps of:
collecting whole blood with or without anticoagulant, e.g. by venipuncture, following which blood is (immediately) centrifuged at speeds of 200-800xg, preferably 400-800xg, more preferably 600-800 xg and most preferably at about 800xg, for 5-20 minutes, more preferably 7.5-15 minutes, e.g. around 10 minutes;
collecting the plasma and isolating P-WBC; and
cryo-preserving the P-WBC by storing them at a temperature of between −60 to −90° C., preferably-70 to −85° C., most preferably at around −80° C., using a cryoprotectant, preferably DMSO, e.g. 6% DMSO.

The cryopreserved samples are suitable for shipment to, e.g., a central laboratory equipped to determine any given biomarker, such as damaged sGC (based on the sGCa/sGCs ratio as defined herein), in a highly standardized and reliable manner. The cryopreserved P-WBC can suitably be stored for a period of at least 3 months before the biomarker is determined. In preferred embodiments of the invention, prior to determination of the biomarker, the cryopreserved P-WBC are thawed at a temperature of about 37° C., for a period of, e.g., about 5 minutes.

### Pharmaceutical Kits

Another aspect of the invention is directed to a pharmaceutical kit comprising a package containing a plurality of unit dosage forms and a leaflet, wherein said unit dosage forms contain a pharmaceutical composition according to the invention and wherein said leaflet contains printed instructions to repeatedly self-administer said unit dosage forms in order to accomplish any of the therapeutic objectives as defined herein.

In accordance with embodiments of the invention, the pharmaceutical kit comprises a container, such as a cardboard box, holding one or more blister packs, said one or more blister packs contain a plurality of solid unit dosage forms as defined herein before. In particularly preferred embodiments of the invention, the pharmaceutical kit comprises at least 5, at least 8, at least 10, at least 12 of at least 15 of said unit dosage forms, e.g. 4, 5, 6, 7, 8, 9, 10, 11, 12, 13, 14, 15, 16, 17, 18, 19 or 20 of said unit dosage forms.

In accordance with the invention, the pharmaceutical kit comprises a leaflet inserted into the container, typically a patient information leaflet containing printed information, which information may include a description of the form and composition of the unit dosage forms contained in the kit, an indication of the therapeutic indications for which the product is intended, instructions as to how the product is to be used and information and warnings concerning adverse effects and contraindications associated with the use. It will be understood by those of average skill in the art, based on the information presented herein, that the leaflet that is part of the kit according to the invention, will typically contain the information concerning the therapeutic indications, uses, treatment regimens, etc. as described here above in relation to the methods of treatment of the present invention. In particularly preferred embodiments of the invention, the leaflet contains printed instructions to repeatedly (self-) administer the unit dosage forms in order to treat and/or prevent HFpEF, in particular in subjects having an impaired NO-cGMP-PKG signaling axis, as defined herein elsewhere.

In one embodiment of the invention, the pharmaceutical kit comprises a plurality of unit dosage forms as defined herein that contain the sGC (positive) modulator, the NO recoupler and the NO substrate (precursor), preferably in the quantities defined herein elsewhere.

In one embodiment of the invention, the pharmaceutical kit comprises a plurality of unit dosage forms as defined herein that contain the sGC (positive) modulator and the NO recoupler, preferably in the quantities defined herein elsewhere as well as a plurality of unit dosage forms as defined herein that contain the NO substrate (precursor), preferably in the quantities defined herein elsewhere.

In one embodiment of the invention, the pharmaceutical kit comprises a plurality of unit dosage forms as defined herein that contain the sGC (positive) modulator and the NO recoupler, preferably in the quantities defined herein elsewhere and the leaflet contains printed instructions to repeatedly (self-) administer the unit dosage forms in combination with treatment with an NO substrate (precursor).

In one embodiment of the invention, the pharmaceutical kit comprises a plurality of unit dosage forms as defined herein that contain the sGC (positive) modulator, preferably in the quantities defined herein elsewhere and the leaflet contains printed instructions to repeatedly (self-) administer the unit dosage forms in combination with treatment with an NO recoupler and/or an NO substrate (precursor).

### Miscellaneous

It is to be understood that many or most of the pharmacologically and/or physiologically active ingredients contained in the compositions as defined herein can exist in the form of a pharmaceutically acceptable salt or solvate. Such forms will typically be equally suitable for use in the present invention.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

“A”, “an”, and “the” as used herein refer to both singular and plural forms unless the context clearly dictates otherwise. By way of example, “a compartment” refers to one or more than one compartment.

“About” as used herein referring to a measurable value such as a parameter, an amount, a temporal duration, and the like, is meant to encompass variations of +/−10% or less, more preferably +/−5% or less, even more preferably +/−1% or less of and from the specified value, in so far such variations are appropriate to perform in the disclosed invention. However, it is to be understood that the value to which the modifier “about” refers is itself also specifically disclosed.

“Comprise”, “comprising”, “comprises” and “comprised of” as used herein are synonymous with “include”, “including”, “includes” or “contain”, “containing”, “contains” and are inclusive or open-ended terms that specify the presence of what follows, e.g. a component, and do not exclude or preclude the presence of additional, non-recited components, features, element, members, steps, known in the art or disclosed therein.

The recitation of numerical ranges by endpoints includes all numbers and fractions subsumed within that range, as well as the recited endpoints. The skilled person will appreciate that the present invention can incorporate any number of the specific features described above.

### DESCRIPTION OF THE FIGURES

FIG. 1 illustrates the healthy (right side) and the pathologically altered pathways in microvascular perfusion. In a healthy situation, nitric oxide synthase (NOS) synthesises NO, which binds to sGC and thereby increases cGMP formation. In a subset of HFpEF cases, NADPH oxidase type 5 (Nox5) creates excess amounts of reactive oxygen species (ROS), which uncouple and deactivate NOS. Separately, ROS deplete NO by converting it to peroxynitrite (ONOO⁻), a reactive nitrogen species that can nitrate proteins, especially their tyrosine residues.

FIG. 2: HFpEF patients with microvascular cardiac hypoperfusion in whom the Nox5/ROS/NOS uncoupling mechanism is operative are identified by determining levels of Nox5 and nitrotyrosine in blood and exosomes. These patients are treated with recoupling agents that restitute the enzymatic activity of NOS, and also receive an agent that stimulates sGC to produce more cGMP from the NO that is now available again.

FIG. 3 is an illustration of Example 1. HFpEF is induced in C57BL/6J wildtype mice by chronic infusion of angiotensin II and (with angiotensin-II treatment continued) mice are randomized into three groups which additionally receive either placebo or triple therapy with the NOS recouplers L-citrulline (500 mg/kg/day) and folate (15 mg/kg/day) plus the sGC modulator vericiguat in a low-(3 mg/kg/day or a high (10 mg/kg/day) dose.

FIG. 4a-c summarise the functional improvement of HFpEF mice observed in Example 1 as seen in echocardiography parameters. Left atrial area (FIG. 4a) and isovolumetric relaxation time (FIG. 4b) significantly increased after chronic infusion of low dose ATII for four weeks, indicating a successful induction of an HFpEF-phenotype in C57BL/6J mice (baseline vs Ang II). After treatment with the triple therapy, including vericiguat in either low dose (3 mg/kg/day; Ang II+low 3T) or high dose (10 mg/kg/day; Ang II+high 3T), left atrial area and isovolumetric relaxation time were significantly reduced indicating a substantial improvement of diastolic function of the left ventricle. Control treatment showed substantial impairment of diastolic dysfunction. Importantly, none of the treatments significantly affected the left ventricular ejection fraction as a sign of systolic function (FIG. 4c). (n=6-8; * p<0.05, ** p<0.01, *** p<0.001, **** p<0.0001, n.s. non-significant)

FIG. 5 illustrates that running wheel activity was significantly improved after triple therapy with L-citrulline, folic acid and either low-dose or high-dose vericiguat (Ang II+low 3 T, Ang II+high 3 T) compared to control (n=6-8; * p<0.05) FIG. 6a-c shows that triple therapy treatment with low-dose or high-dose vericiguat showed no significant changes in systolic, diastolic and mean blood pressure as assessed by a non-invasive tail-cuff blood pressure analyser (n=6-8; n.s. non-important)

FIG. 7 indicates that decline in body weight after the induction of HFpEF with chronic low-dose ATII infusion was ameliorated by high-dose triple therapy (n=6-8; * p<0.05, **** p<0.0001). Hereby, Sham indicates the physiologic progression of the body weight during maturation throughout the course of the experiment without any treatment.

FIG. 8 shows the validation of the NOX5 biomarker for HFpEF patient endotyping. NOX5 plasma levels in biobanked plasma samples from HFpEF patients. Patients were classified as HFpEF according to the HFAPEFF classification score i.e., HFA-PEFF score≥5. FIG. 8a shows the distribution of NOX5 levels in patients stratified according to the HFA-PEFF score. FIG. 8b shows the data from FIG. 8a clustered in 10 ng/ml blocks, revealing a bimodal distribution, with two subgroups (visualized in the graph) in a ratio of 2:1 and an approximate cut-off value of 105 ng/ml.

FIG. 9 shows 3-NT plasma levels in biobanked plasma samples from HFpEF patients. Patients were classified as HFpEF according to the HFA-PEFF classification score i.e., HFA-PEFF score≥5.5 out of 7 patients that had high NOX5 levels (indicated by the dark shaded dots) also show very high levels of plasma 3-NT (>1200 nM).

FIG. 10 displays the endotyping of patients who are preselected by clinical HFpEF scores, by detecting elevated NOX5 or ROS-dependent metabolites and/or impaired cGMP signalling. FIG. 10a represents detection of increased NOX5 protein in plasma/exosomes as stable biomarker of a pathological ROCG module. FIG. 10b shows the collection of white blood cell-containing platelet-rich plasma (wPRP). Both can be utilised to analyse the functional state of the ROCG disease module. Physiological NOS3-derived NO binding to and increasing sGC activity to form cGMP is displayed, which activates cGMP-dependent protein kinase (PKG). In the event of increased formation of reactive oxygen species (ROS, red), e.g. by NOX5, sGC's heme is oxidised and detaches resulting in apo-sGC or less sGC is formed from apo-sGC, both having the same net result that NO formation results in less cGMP. The ratio between sGC and apo-sGC can be analysed by using a sGCs and comparing it to an sGCa using a PKG substrate, e.g. phosphorylated vasodilator-stimulated phosphoprotein, as a read-out. Since the sGC stimulator is also used for intervention to sensitise sGC for lower NO levels, this also represents an ex-vivo drug response test. Also shown is a second consequence of ROS leading to uncoupling of NOS3 (uc-NOS3, in red) involving both an arginine metabolite that competes with the NOS substrate, L-arginine, and oxidation of the NOS cofactor, tetrahydrobiopterin. NOS can be recoupled by offering L-arginine through L-citrulline, which has a higher bioavailability, and folate, regenerating tetrahydrobiopterin through the so-called salvage pathway. Targeting the ROCG disease module of the HFpEF endotype by network pharmacology at two sites (uc-NOS and sGC) induces synergy, allows low or lower than marketed doses of the compounds and lowers the risk of any potential hydrodynamic or other side-effects. FIG. 10c shows a comparison of cryopreserved vs fresh cell preparation of healthy participants by evaluating sGC/apo-sGC ratio by the induced phospho-VASP response to an sGC stimulator and an sGC activator. Different colours depict different biological replicates (n=3). Cryopreserved sample allows the detection of sGC oxidation status, making the assay suitable for use in a clinical setting where patients' samples are cryopreserved and shipped for further analysis.

FIG. 11: First neighbours' protein-protein interaction network of sGC. Soluble guanylate cyclase subunits (GUCY1A1, GUCY1A2, GUCY1B1) were selected as seeds (black dots) and mapped within the human interactome of all experimentally validated protein interactions. Next, their first neighbour proteins (i.e., proteins one interaction away) were identified to extract the ROCG disease module. Here, amongst other proteins, NADPH oxidase 5 (NOX5) was found directly connected to sGC (GUCY1B1) as the only ROS-generating enzyme of this module. The endothelial NO synthase (NOS3) also appeared directly linked to sGC (GUCY1B1). The shortest path of the extracted module to chaperonin containing TCP1 subunit 7 (CCT7) was also identified (black square), but it was found outside the first neighbours' network.

FIG. 12: Different centrifugal forces (g) for P-WBC isolation. P-WBC is isolated after blood centrifugation; upon treatments with sGCs or sGCa, cGMP is produced and activates PKG, which phosphorylates VASP. 12B) Separation of blood sample in different fractions, i.e., plasma, P-WBC, and red blood cells upon a 10 minutes centrifugation at different centrifugal forces (200 g, 400g, 600g, 800g). 12C) Cell recovery of platelets (PLT), white-blood cells (WBC) and red-blood cells (RBC) in P-WBC using different centrifugal speeds. 12D) pVASP/VASP response to an sGCs (BAY 41-2272, 30 μM) divided by the pVASP/VASP response to an sGCa (BAY 58-2667, 10 μM) in P-WBC obtained using different centrifugal speeds. 12E) pVASP/VASP response to an sGC stimulator (BAY 41-2272, 30 M) in P-WBC obtained using different centrifugal speeds. Below: Representative western blot images. (n=4-5, mean±SEM). The white bars represent the most optimal assay conditions.

FIG. 13. Evaluation of different cryopreservation and thawing conditions, and time of storage. P-WBC is isolated after a 800g centrifugation and cryopreserved. Upon thawing, P-WBC is treated with sGCs or sGCa, cGMP is produced and activates PKG, which phosphorylates VASP. 13B,C) P-WBC were cryopreserved using different conditions and stored for 1 week at −80° C. Samples were thawed after exposure to RT for 2 min followed by 1 min at 37° C. and treatments were subsequently performed. pVASP/VASP response to an sGCs (BAY 41-2272, 30 μM) divided by the response to an sGCa (BAY 58-2667, 10 μM) for each condition is depicted (n=5, mean±SEM). 13C) Representative western blot images after cryopreservation using different conditions. 13D) Different thawing methods were tested (2 min-RT & 1 min−37° C., 1 min−37° C., 5 min−37° C.) after cryopreservation of P-WBC for 3 days using 6% DMSO and the induced pVASP/VASP response to an sGCs (BAY 41-2272, 30 μM) divided by the response to an sGCa was measured (n=3, mean±SEM). 13E) pVASP/VASP response to an sGCs (BAY 41-2272, 30 μM) divided by the response to an sGCa (BAY 58-2667, 10 μM) after cryopreservation of P-WBC for 1 or 3 months was measured (n=4, mean±SEM, ns: not significant). These samples were cryopreserved with 6% DMSO and thawed for 5 min−37° C. Below each graph: Representative western blot images. The white bars represent the most optimal assay conditions.

FIG. 14. Concentration response curves of cGMP-related compounds in fresh P-WBC. P-WBC is isolated after a 800g centrifugation and treated with sGCs or sGCa. CGMP is produced and activates PKG, which phosphorylates VASP. IBMX inhibits PDES, which are responsible for cGMP degradation, thereby maintaining the produced cGMP levels. 14B) pVASP/VASP response to different doses of the sGCs (riociguat) or the sGCa (runcaciguat). 14C) Left: Concentration response curves of IBMX in presence of subthreshold doses of sGCs (riociguat 3 μM) or sGCa (runcaciguat 10 nM). The data represent the response to the sGCs or sGCa after subtracting the basal IBMX response. Right: Representative western blots after stimulation with different doses of IBMX alone or in the presence of riociguat (3 μM) or runcaciguat (10 nM). 14D) Concentration response curve of riociguat or runcaciguat in presence of 500 μM IBMX (n=3, mean±SEM). Below each graph: Representative western blot images.

FIG. 15. ROCG mechanism-based biomarkers in HFpEF patients. 15A) Plasma NOX5 levels were measured in HFpEF patients from the Maastricht cohort (classified according to the HFA-PEFF diagnostics algorithm). A plasma NOX5 cut-off value can be observed—in the violin plot (left) and in the histogram (right) graphical representations—at 105 ng/ml, defining the HFpEF NOX5-related endotype (yellow shaded area). 15B) The ROCG module in signalling representation. NOX5 leads to uncoupling of NO synthase (area with////pattern fill). In addition, sGC signalling may be affected by ROS including loss of heme and a shift to apo-sGC (area with \\\\ pattern fill). 15C) pVASP/VASP response to the sGCa (runcaciguat, 1 μM) divided by the response to the sGCs (riociguat, 100 μM) in cryopreserved P-WBC from HFpEF patients from the Valencia cohort. In this cohort, patients were classified as HFpEF according to the REPO-HFpEF inclusion criteria. The data are represented as a frequency histogram. Dashed line on sGCa/sGCs value at 1.05 represents the cut-off, after which the second peak in the distribution appears (area with \\\\ pattern fill). 15D) Plasma NOX5 levels measured in patients from the Valencia cohort also suggested a cutoff value at 105 ng/ml (area with////pattern fill). 15E) Combined plot of NOX5 levels and sGCa/sGCs values in the Valencia-HFpEF cohort (NOX-5 levels from D, and sGCa/sGCs from C). The dashed line on 105 ng/ml on the y-axis represents the NOX5 cut-off and the area above it is marked with the////pattern fill. The dashed line on 1.05 on the x-axis represents the sGCa/sGCs cut-off and the area above it is marked with the \\\\ pattern fill.

FIG. 16. The REPO-HFpEF II trial design. The different columns indicate the different trial phases, mechanism-based screening for the absence or presence of ROCG biomarkers, randomization (verum: placebo, 2:1), treatment and follow-up phase.

FIG. 17. Plasma levels of the ROS biomarker NOX5 in HFpEF patients from the Maastricht cohort. Patients were considered as HFpEF according to the H2FPEF diagnostics score. The data is represented as a violin plot (left) and a histogram (right).

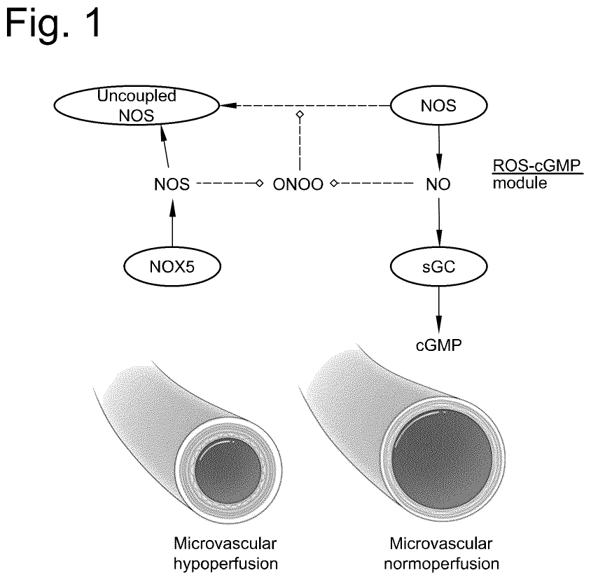

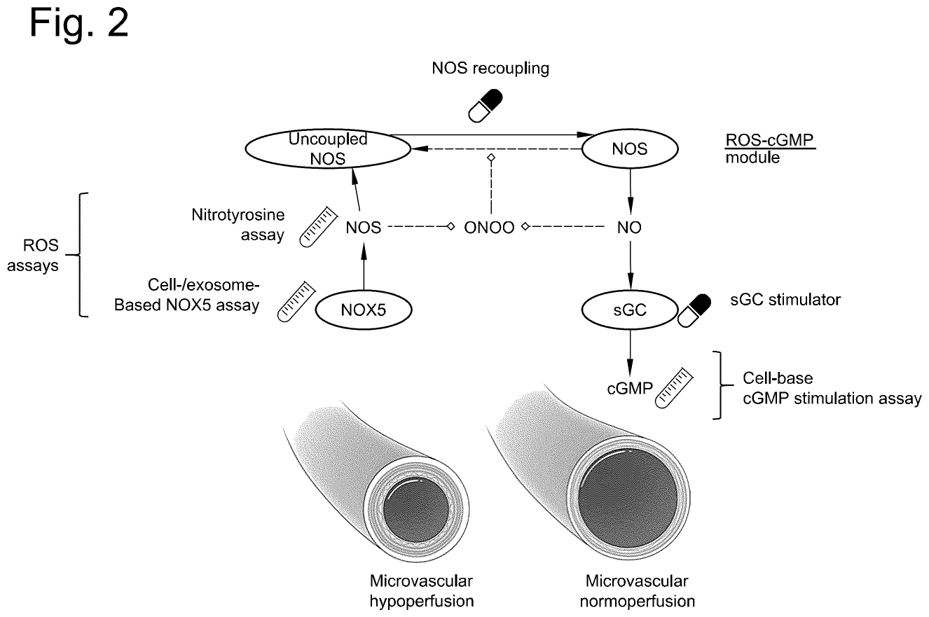

### EXAMPLES/EXPERIMENTAL

### Example 1: Identification of the HFpEF Endotype Characterized by High ROS-Related Biomarkers and Insufficient NO-cGMP-PKG Signalling

Plasma samples from HFpEF patients were collected from the Biobank Maastricht UMC+. Patient were classified as HFpEF if the HFA-PEFF score returned by the HFA-PEFF diagnostic algorithm was ≥5. The HFA-PEFF diagnostic algorithm is a multistep process that considers a patient's echocardiography and natriuretic peptides levels for the diagnosis.

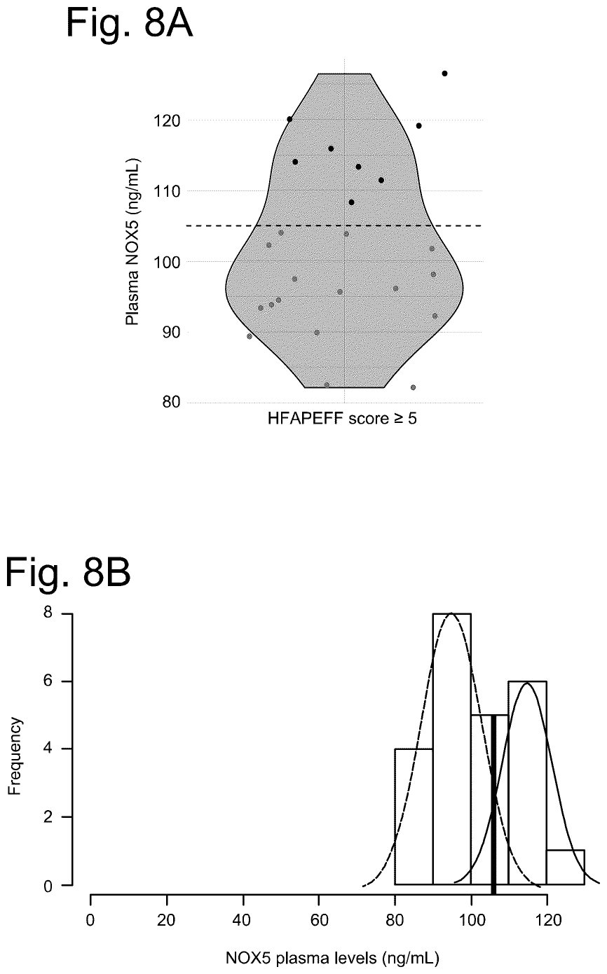

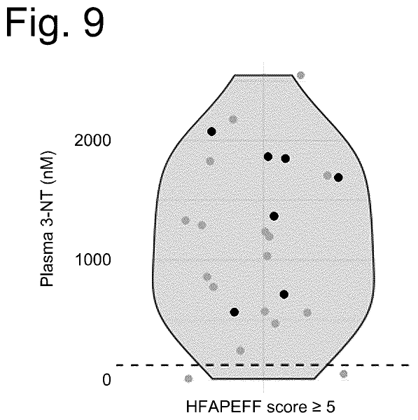

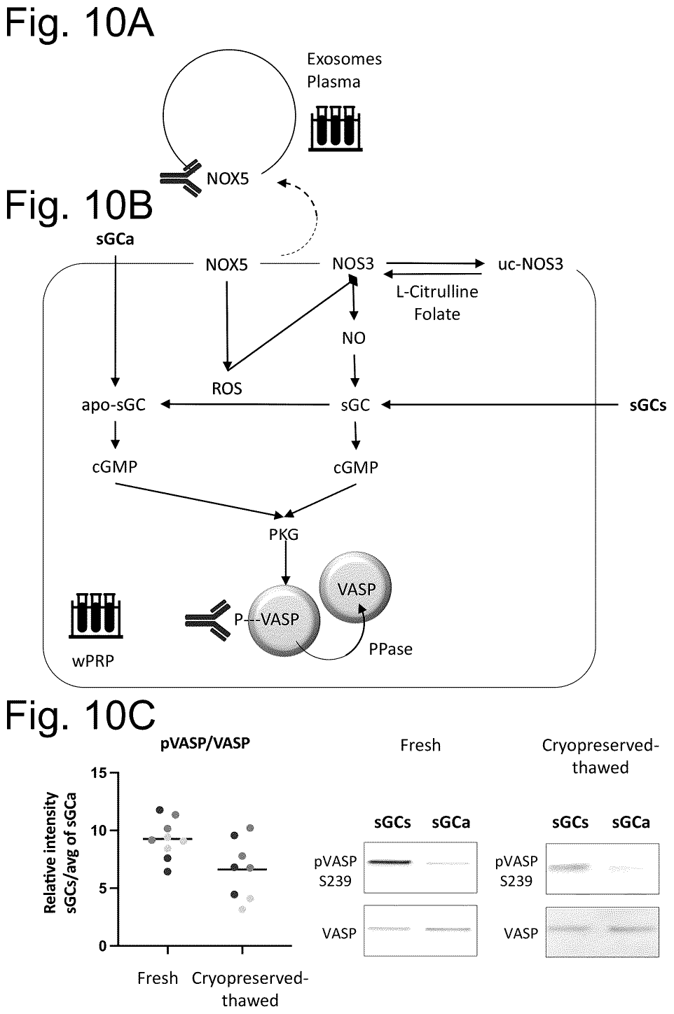

NOX5 plasma levels were measured in these samples using a commercial NOX5 ELISA kit (MyBiosource, #MBS2512643). 3-nitrotyrosine plasma levels were also measured using a 3-nitrotyrosine commercial ELISA kit (Abcam, #ab210603). The results are displayed in FIGS. 8 and 9.

As can be seen in FIG. 8, the biobanked patient population studied separated in two groups according to their NOX5 plasma levels at roughly 110 ng/ml plasma NOX5 (FIG. 8a). Patients with NOX5 plasma levels above this cut-off value (i.e., 110 ng/ml) are potential candidates likely to benefit from the described triple therapy, including vericiguat, L-citrulline and folic acid, and may be further endotyped on the basis of cGMP production and/or the phosphorylation in a sample containing cells and/or exosomes, in response to a stimulus.

### Example 2: Triple Therapy Including Vericiquat, L-Citrulline and Folic Acid in a Mouse Model of Heart Failure with Preserved Ejection Fraction (HFpEF)

The therapeutic efficacy of triple therapy including vericiguat, L-citrulline and folic acid was investigated in a mouse model of heart failure with preserved ejection fraction. In this model, mice are subjected to chronic infusion of a low suppressor dose of angiotensin II. Angiotensin II infusion as used in this model affects ROS production and signalling. The model is therefore considered to represent the HFpEF endotype characterized by insufficient NO-cGMP-PKG signalling.

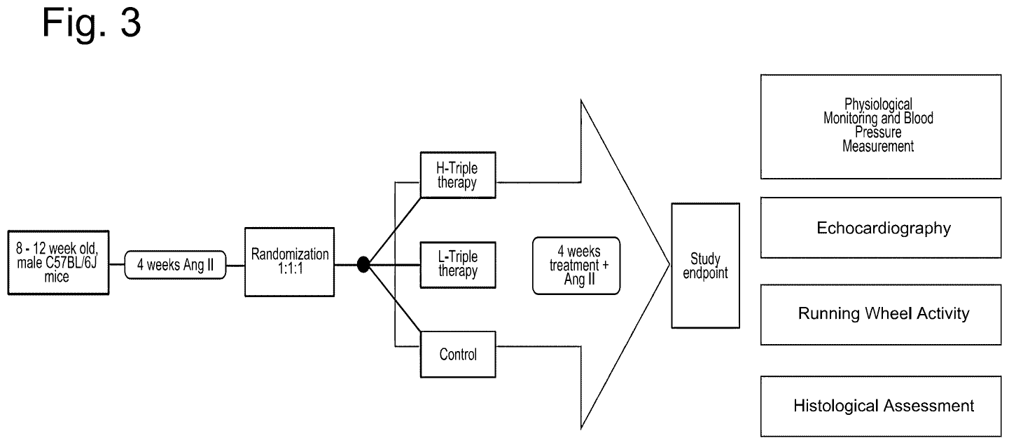

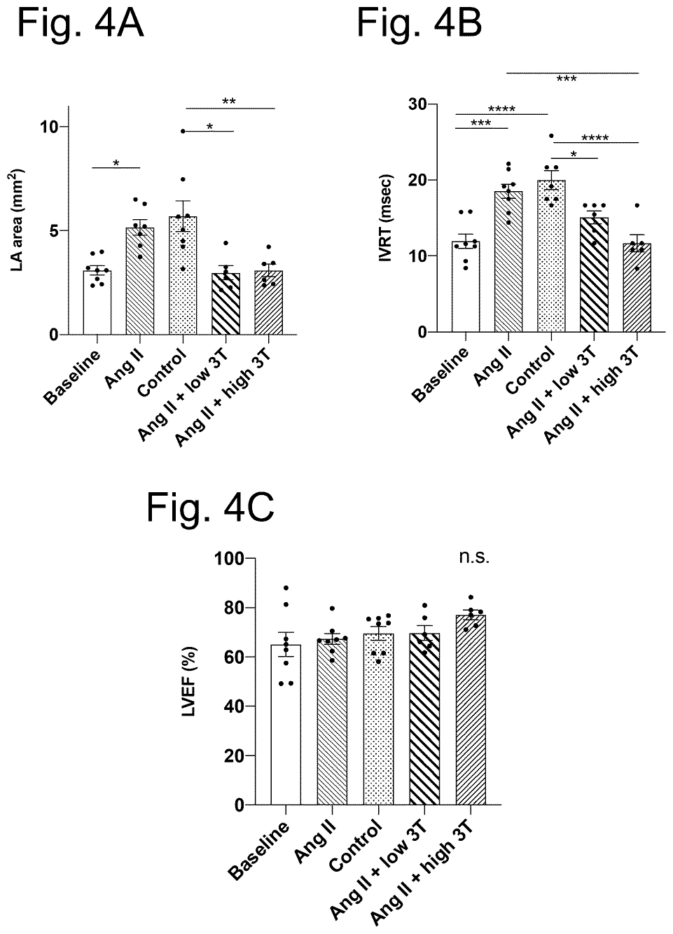

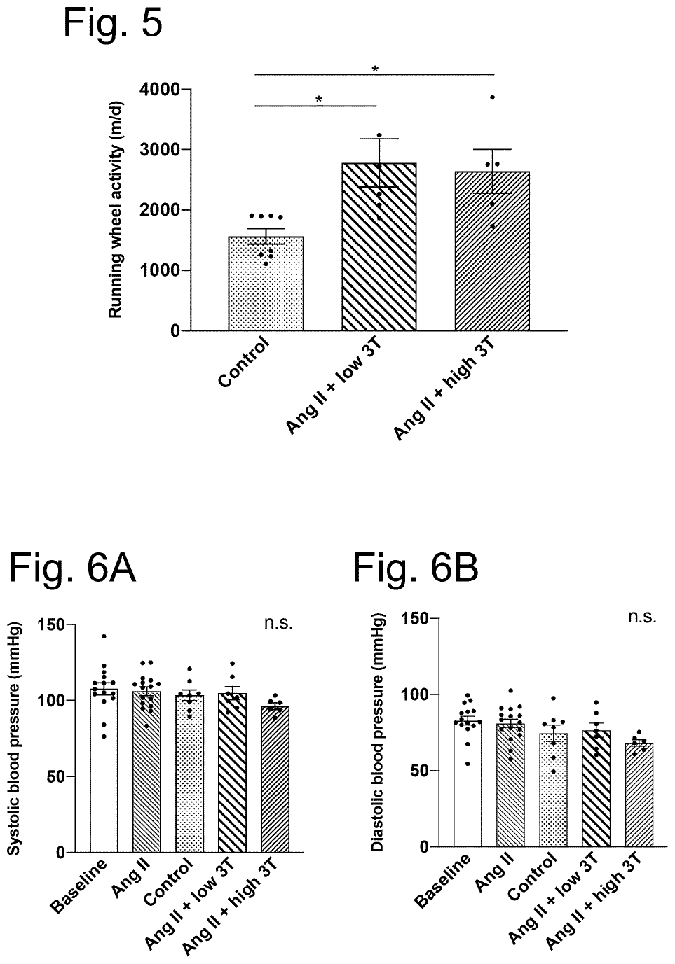

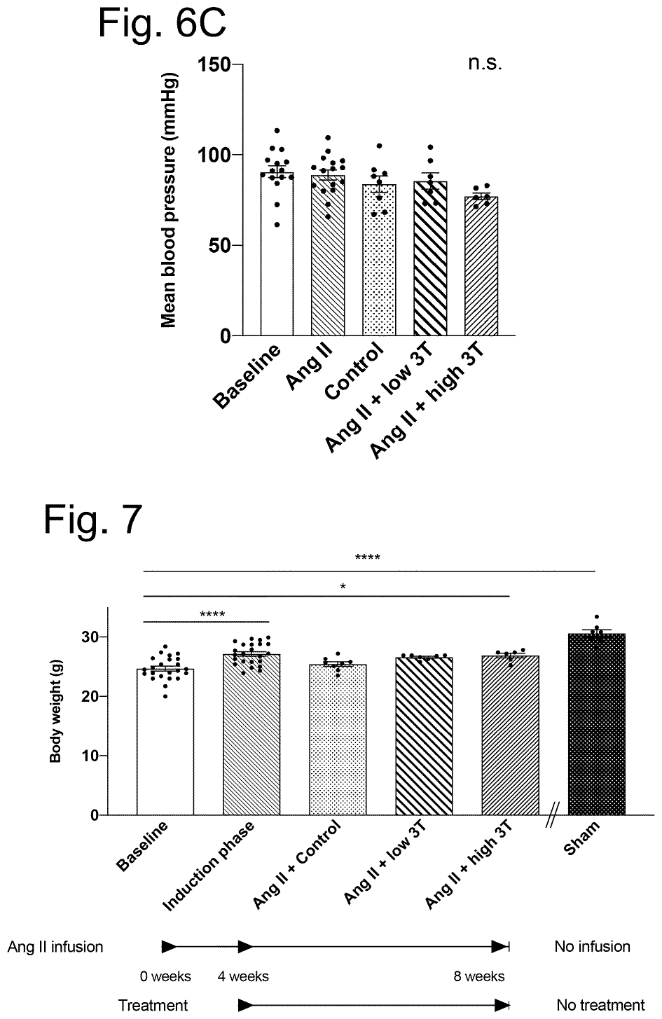

An osmotic micropump (Alzet, Model 1004; DURECT Corporation) was subcutaneously implanted in the flank of 8 to 12-week-old male C57BL/6J wild-type mice via a small incision. Low-dose angiotensin II (AT-II; 0.2 mg/kg/day; A9525, Sigma-Aldrich) or physiological saline solution was chronically infused via the micropump for 28 days to either induce HFpEF or serve as a control, respectively. After 28 days, mice were assessed for physical parameters, and hearts are investigated by echocardiography and histology (FIG. 3). Left atrial area (FIG. 4a) and isovolumetric relaxation time (FIG. 4b) had significantly increased while ejection fraction remained essentially unchanged (FIG. 4c), indicating the onset of HFpEF. Micropumps were replaced to maintain chronic infusion treatment of either AT-II or physiological saline.

Concurrently, AT-II infused mice were randomised to treatment groups to receive a combination of L-citrulline (500 mg/kg/day) and folic acid (15 mg/kg/day) together with vericiguat in either a low dose 3 mg/kg/day) or a high dose (10 mg/kg/day) by oral gavage for 28 days after HFpEF induction. Systolic and diastolic arterial blood pressure was non-invasively measured in a conscious state in all mice at baseline, at 28 days and at 56 days using a tail-cuff blood pressure analyser (CODA System, Kent Scientific, Torrington, CT),

On day 50, mice were placed individually in cages equipped with a running wheel connected to a data acquisition unit. Mouse health and functionality of the running wheels were controlled daily. On the final day, mice were anaesthetised with isoflurane, and echocardiographic analysis was performed in the supine position. Parameters of systolic and diastolic function were acquired (e.g. LVEF, PW-Doppler flow profiles, left atrial area, isovolumic relaxation time, and early filling deceleration time). Assessment of diastolic function in mice was performed using a published algorithm (Schnelle et al., J. Mol. Cellular Cardiol. 114 (2018) 20-28). Following echocardiographic assessment, mice were sacrificed and tissue was collected for histological analysis.

Both triple therapies significantly reduced left atrial area and isovolumetric relaxation time, indicating a substantial improvement of the left ventricle's diastolic function, while control treatment showed substantial impairment of diastolic dysfunction (FIG. 4a-c). Importantly, none of the treatments significantly affected the left ventricular ejection fraction as a sign of systolic function (FIG. 4c). Triple therapies did not induce significant systolic, diastolic and mean blood pressure (n=6-8; n.s. non-significant) (FIG. 6a-c). Running wheel activity was significantly improved after both triple therapies (n=6-8; p<0.05)

### Example 3: Clinical Study with Triple Therapy (Vericiquat, L-Citrulline and Folic Acid) in HFpEF Patients with the Nox5 Overexpression Endotype of HFpEF

Approximately 100 HFpEF patients>45 yrs of age diagnosed according to the HFA-PEFF algorithm (NYHA classes II-IV, left ventricular ejection fraction at least 50%, elevated NT-proBNP levels, echocardiographic evidence of structural heart disease) are screened for Nox5 overexpression by ELISA. A Nox5 level of ≥110 ng/ml (‘Nox5 overexpression endotype’) is considered characteristic of impaired NO-CGMP-PKG axis.

Approximately 20 Nox5 overexpression endotype patients are randomised to receive for 12 weeks either a triple therapy consisting of folate, L-citrulline, and vericiguat on top of standard of care; or only standard of care. Safety parameters for the triple combination, as measured by adverse drug reactions possibly or definitely related to the investigational agents, will constitute the primary endpoint. Secondary endpoints are change in KCCQ score, NYHA functional class, change from baseline of echocardiography parameters, 6-minute walking distance, and N-terminal pro-BNP An interim analysis is performed for primary and secondary parameters once 6-10 patients have completed the study.

### Example 4: Diagnosing cGMPopathy for Mechanism-Based Intervention in a Subtype of Heart Failure with Preserved Ejection Fraction

### I. Introduction/Background

A mechanism-based diagnostic assay could have prevented such failures, enabling mechanistic endo/subtyping and patient stratification. No such assay is currently available and measuring plasma cGMP levels, most of which is derived from natriuretic peptide signalling, turned out to be futile to measure sGC related drug responses. Other cGMP-related biomarkers mainly include natriuretic peptide levels for HF diagnosis and, phosphorylated vasodilator-stimulated phosphoprotein (P-VASP), a target of cGMP-dependent protein kinase, proposed for antiplatelet therapy guidance. However, because of fast dephosphorylation kinetics, the most reliable way to measure P-VASP is inducing its phosphorylation ex vivo, not a practical approach in a clinical or point-of-care setting.

It was therefore aimed to develop a simple, point-of-care compatible workflow that includes cell-based analysis of cGMP signalling. First, in healthy individuals, it was tested whether isolation of the platelet-enriched white-blood-cell fraction (P-WBC) from a whole-blood sample, followed by cryopreservation and shipment to a specialised lab allowed for the detection of cGMP signalling and this with minimal effort required at the clinical site. The best indication for ROS affected sGC is an elevated ratio of the oxidatively damaged, heme-free apo-form of sGC versus the healthy heme-containing form. Both states can be probed by using sGC activators and sGC stimulators, respectively and P-VASP protein immunoblotting as read-out. With respect to the possible disease triggering source of ROS, NOX5 has recently been identified as the closest protein neighbour. Thus, its alternative or additional relation to a cGMP-related HFpEF subtype was investigated in parallel. Then detection of NOX5 levels in plasma by ELISA was applied to biobanked samples from HFpEF patients (Maastricht HFpEF cohort), selected either based on the definition of the European Society of Cardiology or the American Heart Association. Then, with both assays, samples from patients considered for the mechanism-based REPO-HFpEF II trial, which will investigate the effect of sGC stimulators in combination with NO synthase recoupling in ROCG-positive HFpEF patients, were analysed.

### II. Materials and Methods

### Chemicals

ACD-A tubes were obtained from Fisher Scientific (BD 366645). Riociguat (BAY 63-2521) and runcaciguat (BAY 110-1042) were obtained from MedChemExpress and IBMX from Enzo. BAY 58-2667 was from Merck and BAY 41-2772 was from Enzo. Pierce Phosphatase Inhibitor cocktail was from ThermoFischer Scientific and complete (TM), Mini Protease Inhibitor Cocktail from Merck. ROTI®Load 1 buffer was obtained from CarlRoth. Anti-phosphoVASP Ser 239 (clone 16C2) antibody was purchased from Nanotools (0047-100) and total VASP polyclonal antibody (ALX-210-898) was from Enzo. HRP conjugated goat anti-rabbit and rabbit anti-mouse antibodies were from Agilent Dako. DMSO and trehalose were from Sigma. NOX5 Elisa was from MyBioSource (MBS2512643).

### Protein-Protein Interaction Module

The NeDRex Cytoscape plugin for network medicine was used to explore the protein neighbours of soluble guanylate cyclase (sGC) and extract the relevant disease module. All sGC subunits from UniProt (GUCY1A1, GUCY1A1 and GUCY1B1) were considered and used as seeds to start building the network. GUCY1B2 has no experimentally known human protein interactions in IID and was thus not considered. First neighbour interacting proteins were added next resulting in the final protein-protein interaction network. To explore the potential link of this network to the recently described chaperonin containing TCP1 subunit 7 (CCT7), the shortest path to the network was computed with a Steiner tree.

P-WBC Preparation for cGMP-Assay Optimisation

Human blood was collected from healthy volunteers after obtaining informed consent according to the Declaration of Helsinki. An ethical approval was granted by the FHML Research Ethics Committee of Maastricht University (FHML-REC/2022/055). Whole blood (WB) was collected by venipuncture in ACD-A tubes (sodium citrate: 22.0 g/L, dextrose 24.5 g/L, citric acid: 8.0 g/L). Centrifugations in various speeds (200 g, 400g, 600g, 800g) were performed for 10 minutes in 18 degrees Celsius (acceleration 1, deceleration 0). Plasma was collected and P-WBC was carefully isolated (500 μl). Cell counting was performed with the automated haematology analyzer Sysmex XP-300. Subsequently, P-WBC was diluted with the same volume of JNL-buffer (130 mM NaCl, 3 mM KCl, 9 mM NaHCO₃, 0.81 mM KH2PO4, 0.9 mM MgCI2, 10 mM sodium citrate, 6 mM dextrose, 10 mM Tris base, 2 mM HEPES) pH 7.4. Thereafter, 20 μl of diluted P-WBC were further diluted 1:5 with JNL-buffer for the cell treatments. Cell suspensions were treated with BAY 41-2272 (30 μM) and BAY 58-2667 (10 μM) for 10 minutes at 37° C., and with riociguat and runcaciguat for 15 minutes; in some cases with 10 minutes pretreatment with IBMX. Samples were centrifuged for 10 minutes at 750 g. Cell pellets were lysed with ROTI®Load Laemmli containing SDS (approx. 2% w/v), β-mercapto ethanol (approx. 5% v/v), glycerol (approx. 10% v/v) and supplemented withphosphatase and protease inhibitors. Lysates were boiled for 5 minutes at 95° C. and stored at −20° C.

Cryopreservation for cGMP-Assay Optimisation

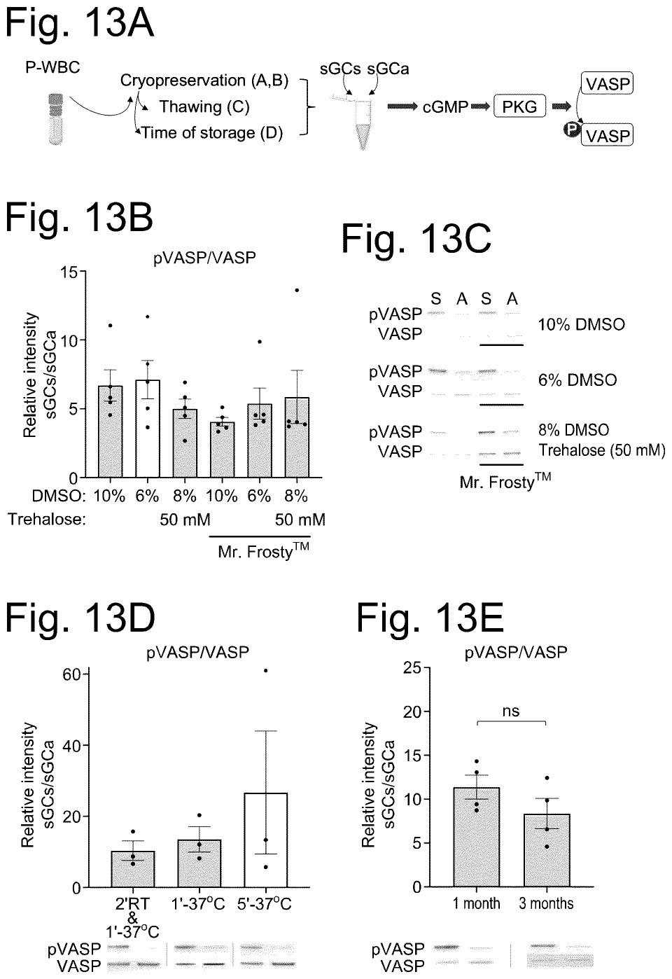

P-WBC was cryopreserved at −80° C. using cryopreserving reagents (DMSO, trehalose) as described in the results (FIG. 13). Upon thawing, samples were diluted 1:100 with JNL-buffer to reduce the cryoprotectant concentration below 0.1% and treated with an sGC stimulator (BAY 41-2272, 30 μM) or an sGC activator (BAY 58-2667, 10 μM). Cell lysates were prepared as described above and the induced phosphorylation-response of VASP was measured by protein immunoblotting.

### Western Blot Analysis

Cell lysates were separated by SDS-PAGE using 8% or 10% Bis-Tris gels and transferred to PVDF membranes. Membranes were stained with Ponceau-S and blocked with 3% non-fat dry milk in TBS-Tween (0.1%) for 1 hour at room temperature. Primary antibody incubation was performed overnight at 4° C. (Anti-phosphoVASP Ser 239 1:400, total VASP 1:2000). Membranes were washed 3 times for 10 minutes with TBS-Tween (0.1%). Rabbit anti-mouse or goat anti-rabbit secondary antibodies were used accordingly for 1 hour at room temperature. Stripping of membranes for reblotting with total antibody was done with boiled solution of glycine 100 mM, pH 2, 3 times for 7 minutes. ECL detection (Amersham) was performed for signal visualisation and densitometric analysis was done with the iBright Analysis Software.

### HFpEF Samples—Maastricht Cohort

Blood has been collected for biobanking from HFpEF patients with informed consent after obtaining approval (NL67997.068.18 and NL76585.068.21) from the Medical Review Ethics Committee of the University Hospital Maastricht and Maastricht university (METC azM/UM). After venipuncture collection, blood tubes were centrifuged at 2000 g for 10 min and plasma was collected and stored at −80° C. Thereafter, NOX5 measurements were performed in plasma samples.

### HFpEF Samples—Valencia Cohort

Once the cGMP-assay was established in principle, blood was taken from twenty HFpEF patients with informed consent after obtaining approval from the Ethics Committee for Research with Medicines of the Hospital Clínico Universitario de Valencia (2022/248) at INCLIVA, Valencia. HFpEF patients were selected based on the inclusion criteria of the planned REPO-HFpEF II trial. After centrifugation at 800g for 10 minutes, 1.5 ml of plasma was collected, aliquoted and frozen at −80° C. P-WBC was isolated, cryopreserved with 6% DMSO and stored at −80° C. NOX5 measurements were performed in the plasma samples. One aliquot of cryopreserved P-WBC (125 μl) was thawed for 5 minutes at 37° C., diluted 1:100 with JNL-buffer and split in 9 conditions. Cell treatments were performed with Runcaciguat (1 μM) and Riociguat (100 μM) at 37° C. for 15 minutes after pre-treatment with IBMX (500 μM, 10 minutes). Subsequently, cell lysates were separated by SDS-PAGE and analysed by

Western Blot as described above.

### NADPH Oxidase 5 Measurements

Levels of the reactive oxygen species (ROS)-forming enzyme, NADPH oxidase 5 (NOX5) were measured in plasma from HFpEF patients by ELISA according to the guidelines of the manufacturer (MyBioSource; MBS2512643, sensitivity: 18.75 pg/mL, detection range: 31.25-2000 μg/mL, coefficient of variation is <10%). Plasma samples were thawed on ice and diluted according to prior measurements. The enzyme-substrate reaction is terminated by the addition of a stop solution and the colour turns yellow. The optical density (OD) is measured spectrophotometrically at a wavelength of 450 nm. The OD value is proportional to the concentration of Human NOX5.

### Statistical Analysis

All data are expressed as mean±standard error of mean (SEM) of at least three independent experiments. Analyses and curve fitting were performed with GraphPad Prism 9.0 (GraphPad Software, San Diego, USA) or in RStudio (Posit team (2022). RStudio: Integrated Development Environment for R. Posit Software, PBC, Boston, MA. URL http://www.posit.co/.). For the dose response curves, a non-linear least squares regression model was used. For comparisons, unpaired Student's t-test was performed and p-value was considered significant.

### III. Results

### An Interactome-Based Disease Network for Soluble Guanylate Cyclase

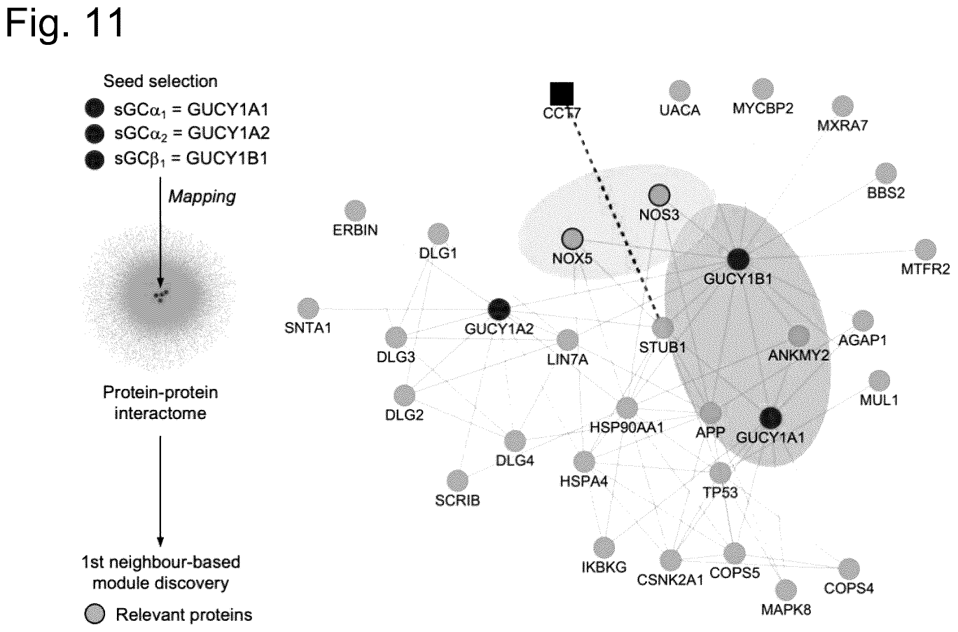

To define the signalling module for the proposed underlying causal mechanism, a first neighbour protein-protein interaction (PPI) network was built starting from all subunits of the soluble guanylate cyclase (sGC) enzyme, i.e., GUCY1A1, GUCY1A2, and GUCY1B1, using the NeDRex platform for network medicine. These subunits were selected as seeds and mapped to the protein-protein interactome, where their first neighbour interactions were extracted to obtain a PPI network with 31 proteins and 81 interactions (FIG. 11). Notably, the reactive oxygen species (ROS)-forming enzyme, NADPH oxidase 5 (NOX5) and endothelial NO synthase (NOS3) were found directly connected to sGC, specifically to the subunit GUCYB1. To explore the recent link observed between the chaperonin containing TCP1 subunit 7 (CCT7) and cardiovascular disease, the shortest path of CCT7 to the sGC network was computed. CCT7 was thus only indirectly connected to the network through STIP1 Homology And U-Box Containing Protein 1 (STUB1).

Development of cGMPopathy Diagnostic Assay

To address current challenges in the diagnostics field of cGMP endotyping, a diagnostic assay was developed that allows for simple and quick patient testing for dysfunctional cGMP signalling. The established protocol includes a blood collection from patients suspected to be suffering from a cGMPopathy, a 1-step quick centrifugation, isolation of P-WBC, and cryopreservation of the sample. Subsequently, samples are shipped to a specialised laboratory, where upon arrival they can be thawed and analysed in order to detect the cGMP endotype among a relevant patient population. To establish the diagnostic protocol, optimisations were performed in which different conditions were evaluated regarding i) centrifugation, ii) cryopreservation, thawing and period of storage, and iii) drugs targeting the cGMP pathway (sGC stimulators and activators).

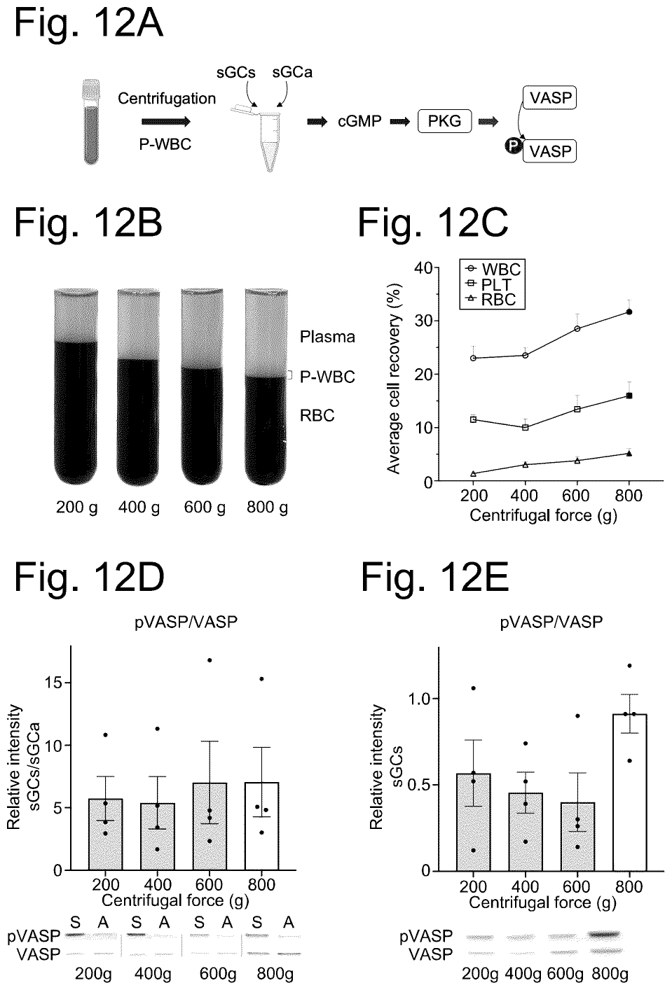

i) Optimisation of centrifugal force: WB was centrifuged at different centrifugal forces (200 g, 400g, 600g, 800g) for 10 minutes. Centrifugal speeds higher than 800 g were not chosen to ensure platelet integrity, and centrifugations longer than 10 minutes were not tested to achieve time efficiency. The sample was separated in three fractions; i.e., plasma, P-WBC and red blood cells (FIG. 12B). Cell counting was performed in P-WBC. The cell recovery was calculated as: cell recovery (%)=[number of cells in P-WBC/number of cells in whole blood]×100. The average cell recovery of both platelets and white blood cells picked at 800 g (16% and 32% respectively) (FIG. 12C). Using an sGC stimulator (sGCs) and an sGC activator (sGCa), which targets the damaged form of sGC, the sGC/apo-sGC ratio was determined via the downstream phospho-VASP (pVASP) response. The treatments with the sGCs or sGCa were performed in the P-WBC. The ratio of the pVASP response to the sGCs divided by the response to the sGCa was similar for the 200, 400 but increased for 600 g and 800 g (FIG. 12D). Comparing only the responses to the sGCs, surprisingly the pVASP signal picked at 800 g (FIG. 12E). Thus, 800 g was selected as the best centrifugal speed from then onwards.

ii) Optimisation of cryopreserving, thawing and storing conditions: Next, different cryopreserving conditions were evaluated, i.e. using DMSO alone as the cryoprotectant at different concentrations, or in combination with the cryoprotectant trehalose, with and without the use of a freezing container (Mr. Frosty TM, cooling rate close to −1° C./minute). The ratio of sGCs- and sGCa-induced pVASP response was tested upon thawing of samples cryopreserved for one week. The induced response using 6% DMSO without a freezing container yielded the highest signal among the tested conditions, showing the most protective effect upon cryopreservation (FIG. 13B,C). Besides the conditions described in the figure, 2.5% DMSO+30 mM trehalose with and without controlled freezing rate was also tested which gave very weak intensity signals, suggesting that cell survival was really low in this case (unquantifiable data, not shown). Thus, 6% DMSO without a controlled freezing container was chosen as the cryopreserving condition. Then, different thawing methods were tested for best recovery after cryopreservation. Using the ratio of sGCs- and sGCa-induced pVASP response, 5 min at 37° C. gave the highest response and was chosen as the preferred thawing method from then onwards (FIG. 13D). Subsequently, the period for which the samples can be stored frozen at −80° C. was assessed. There was not a statistically significant difference between 1 and 3 months in the pVASP response induced by the sGCs and divided by the response induced by the sGCa (FIG. 13E). Thus, cryopreserved samples can be analysed within at least 3 months of storage, which provides enough time for collections, shipments and analyses. In addition, when used for endotype selection in a clinical trial, it is highly impractical and thus unlikely that the samples would have to be stored for more than 3 months.

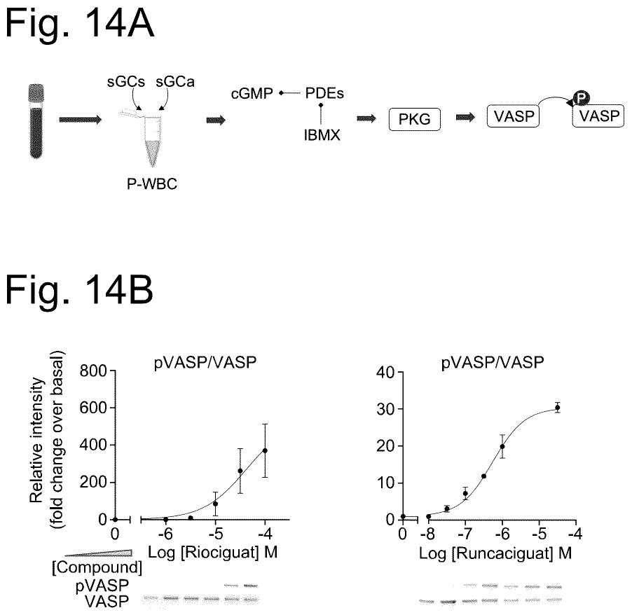

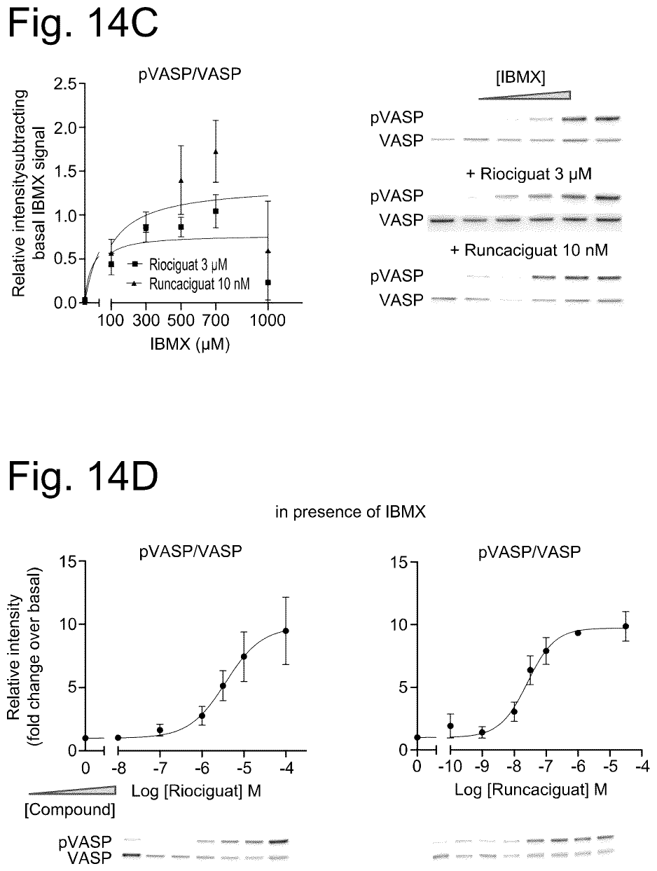

iii) Validation of sGC-targeting compounds and optimisation: In order to validate the sGC oxidation status in patients' samples, it was necessary to select the most suitable concentration for both the sGCs and the sGCa. Clinically relevant drugs were focused on, since the assay could be considered an ex vivo pre-test of the patient's response to the treatment. Riociguat was chosen because it is a sGCs already available in the clinic. At the moment, there is no approved sGCa so runcaciguat was tested, which is currently investigated for diabetic retinopathy (NCT04722991) and chronic kidney disease (NCT04507061). The pVASP concentration response curves to riociguat did not reach a maximum effect even though doses as high as 100 μM were tested, leading to an incomplete curve. However, runcaciguat showed a maximum effect at 100 μM (FIG. 14B). Thus, it was decided to preincubate samples with IBMX in order to inhibit phosphodiesterases' (PDEs) activity. The pVASP response to different IBMX concentrations in the presence of subthreshold doses of riociguat or runcaciguat showed that an IBMX-dose between 500-700 M is the best to potentiate but not overlay the signal induced by the tested compounds (FIG. 14C). Thereafter, the riociguat and runcaciguat concentration response curves were repeated in the presence of 500 μM IBMX. Riociguat's response peaked at 100 μM and runcaciguat's at 1 μM, meaning these are the optimal doses to fully activate sGC and apo-sGC, and evaluate its oxidation status (FIG. 14D).

### ROCG Subtype Identification in HFpEF

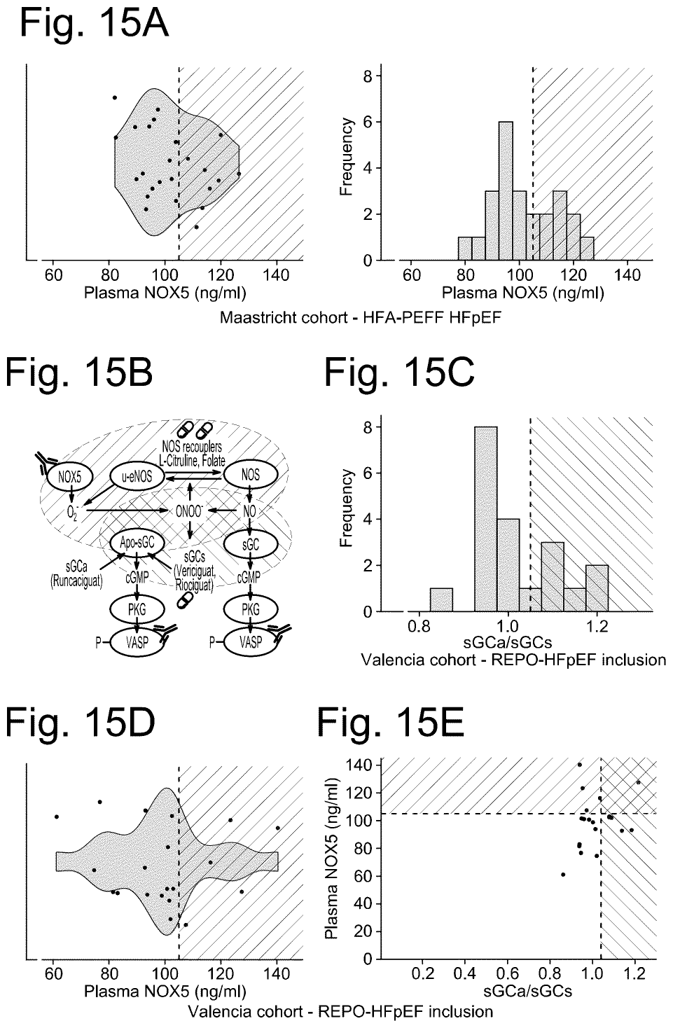

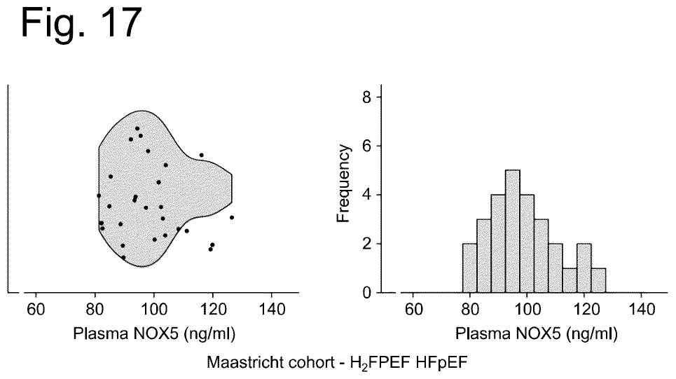

Since NOX5 was found directly connected to sGC in the network (FIG. 11) and has also been previously linked to an endotype of hypertension, plasma NOX5 levels were compared in HFpEF patients from the Maastricht UMC+Biobank either classified by the HFA-PEFF (FIG. 15A) or the H2FPEF score (FIG. 17). Using the HFA-PEFF score, a subgroup of patients (33%; 8 out of 24) was identified with higher NOX5 levels (>105 ng/ml) than the rest, suggesting NOX5 as a potential mechanism-based biomarker (FIG. 15A). Moreover, in a HFpEF cohort from Valencia (which was selected based on the REPO-HFpEF inclusion criteria), the status of damaged/healthy sGC, i.e., apo-sGC/sGC ratio as demonstrated by the pVASP response induced by the sGCa and divided by the response induced by the sGCs, was evaluated. a subgroup of patients (30%; 6 out of 20) was observed with higher sGCa/sGCs values (higher than 1.05), meaning that in those patients the apo-sGC form of the enzyme is more prominent (FIG. 15C). In parallel, when NOX5 plasma levels were measured in the Valencia cohort, a subgroup (25%; 5 out of 20) reappeared based on the NOX5 cutoff value previously described, suggesting to be of significance for the pathology of the disease (FIG. 15D). All together, these two cutoff values i.e., (i) NOX5 plasma levels higher than 105 ng/ml and (ii) an sGCa/sGCs ratio higher than 1.05 (FIG. 15E) mostly do not overlap in the same patients (only in 1 out of 20), showing that both biomarkers are necessary to identify all ROCG module pathomechanisms.

### IV Discussion

A simple, point-of-care compatible diagnostic workflow to detect a dysregulation of the ROCG signalling module in HFpEF patients was developed. It includes a combination of a cell-based analysis of cGMP signalling, i.e. the ratio of apo-sGC and sGC in P-WBC, and the simpler determination of NOX5 plasma levels. When applying this to HFpEF patients selected on the basis of the two leading definitions of the learned societies, ESC and AHA, ESC's HFA-PEFF score appeared superior. This may be because the AHA H2FPEF score has a strong bias for atrial fibrillation. Most noteworthy, clinical trials have not adhered to any of these definitions and used independent, albeit overlapping scores. In preparation of the REPO-HFpEF II trial, a more clinically feasible score was applied that does, however, not require extensive invasive diagnostics. In any case, with the European Union's Horizon Europe REPO-TRIAL project (repo-trial.eu) and the REPO4EU platform (repo4.eu), it is the goal to overcome such symptomatic-phenotypic disease definitions and define patients based on underlying causal mechanisms. When applying ROCG module diagnostics to a cohort of patients selected for screening according to the REPO-HFpEF II protocol, about half of them were ROCG-positive; a quarter for NOX5; another quarter for sGC dysfunction; and one of 20 patients for both.

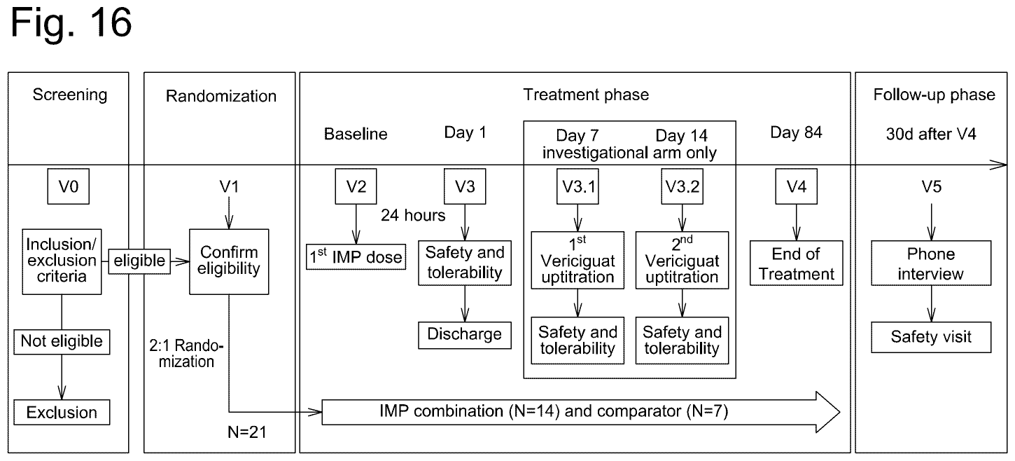

With this mechanism-based stratification, the investigator-initiated REPO-HFpEF II study is designed as a safety, phase IIa, proof-of-concept, unicenter, prospective randomised, standard treatment-controlled, open-label clinical trial (FIG. 16). In addition to the best standard of care, ROCG-positive (NOX5, sGC signalling) HFpEF patients will receive a combination therapy of the sGC stimulator, vericiguat (2.5, 5 up to 10 mg), and the NO synthase recouplers, L-citrulline (3 g) plus folate (5 mg)—NOS3 was directly connected to sGC through protein-protein interactions. Twenty one patients will be randomly assigned on a 2:1 basis, in an open-label, triple therapy or standard of care, which will be given daily over a 12 weeks period. At the end of the treatment period clinical study endpoints (see supplement) will be evaluated. In addition, blood will be drawn and a comprehensive panel of 630 metabolites will be assessed (M×P Quant 500, Biocrates). The primary objective of this study is to assess the safety profile of this mechanism-based triple therapy. Secondary objectives are to investigate possible benefits of treatment on patients, reported outcomes, maximal functional capacity, and echocardiographic and laboratory findings. Based on the strict inclusion and exclusion criteria, it is expected all recruited HFpEF patients will benefit from the cardioprotective effects of this approach by having better outcomes. An adequate safety profile is expected as well as a significant improvement in hemodynamics as measured by echocardiography, exercise tolerance as measured by peak oxygen consumption (peak VO2) assessed by CPET and quality of life as measured by the Kansas City Cardiomyopathy Questionnaire (KCCQ).

With this stratification and revised therapeutic scheme, HFpEF therapy using sGC stimulators such as vericiguat may become effective for at least two reasons: (1) sGC stimulation is combined with NO synthase recoupling, thereby indirectly curing also NOX5-induced NO synthase uncoupling within the ROCG disease module; (2) only those HFpEF patients are treated that are ROCG-positive. Previous trials using sGC stimulators are likely to have attempted to treat a patient group of which half was ROCG-negative and with sGC stimulators alone, NOX5-dependent NO synthase uncoupling remained untreated. Hence only one fourth of the patients may have had a chance to respond leading to a significant dilution of effectiveness.

1.-17. (canceled)

18. Method of prophylactic and/or therapeutic treatment of a subject suffering from HFpEF or at risk of suffering from HFpEF, said method comprising the administration, to said subject, of a composition comprising an sGC (positive) modulator, preferably an sGC stimulator or an sGC activator, characterized in that the subject has an impaired NO-cGMP-PKG signalling axis, wherein impairment in the NO-CGMP-PKG axis functioning is established relying on one or more biomarker based criteria.

19. Method according to claim 18, wherein the subject to be treated meets one or both of the following biomarker based criteria:
a NOX5 plasma level of at least 105 ng/ml; and
an sGCa/sGCs ratio of at least 1.05, wherein the sGCa/sGCs ratio is a value determined by performing assays wherein, in a P-WBC sample obtained from the subject, the pVASP response (pVASP/VASP) to 1 μM of the sGCa runcaciguat, in the presence of 500 μM IBMX (3-isobutyl-1-methylxanthine) is determined, as well as the pVASP response (pVASP/VASP) to 100 μM of the sGCs riociguat (an sGCs), in the presence of 500 μM IBMX, and the sGCa/sGCs ratio is calculated by dividing the response to the sGCa by the response to the sGCs

20. Method according to claim 119, wherein the subject to be treated has a NOX5 plasma level of at least 105 ng/ml.

21. Method according to claim 20, wherein the sGC (positive) modulator is an sGC stimulator or an sGC activator, preferably an sGC stimulator or an sGC activator selected from the group consisting of riociguat, vericiguat, ataciguat, neliciguat, etriciguat, lificiguat, IW-1701, IW-1973, IWP-051, IWP-121, IWP-427, IWP-953, BAY-60-2770, A-344905, A-350619, A-778935, BI-684067, BI-703704, BAY-41-2272, BAY-41-8543, BAY 60-4552, CF-1571, cinaciguat and HMR-1766.

22. Method according to claim 20, wherein the method of treatment further comprises the administration to said subject of an NO recoupler, preferably an NO recoupler selected from the group consisting of folic acid and folic acid salts.

23. Method according to claim 20, wherein the method of treatment further comprises the administration to said subject of an NO substrate or an NO substrate precursor, preferably an NO substrate or an NO substrate precursor selected from the group consisting of L-Arginine, L-citrulline, salts thereof, hydrates thereof, solvates thereof and combinations thereof.

24. Method according to claim 20, wherein the sGC positive modulator is vericiguat and the treatment comprises the administration of vericiguat at a daily dose within the range of 1 mg-50 mg.

25. Method according to claim 20, wherein the sGC positive modulator is riociguat and the treatment comprises the administration of riociguat at a daily dose within the range of 0.2 mg-25 mg.

26. Method according to claim 23, wherein the NO substrate (precursor) is L-citrulline and the treatment comprises the administration of L-citrulline at a daily dose within the range of 0.5 mg-25 mg.

27. Method according to claim 20, wherein the subject has a NOX5 plasma level of at least 110 ng/mL.

28. Method according to claim 20, wherein the pharmaceutical composition further comprises an NO recoupler and/or an NO substrate (precursor).

29. Pharmaceutical composition comprising:
i) an sGC (positive) modulator, preferably an sGC stimulator or an sGC activator, more preferably an sGC stimulator or an sGC activator selected from the group consisting of riociguat, vericiguat, ataciguat, neliciguat, etriciguat, lificiguat, IW-1701, IW-1973, IWP-051, IWP-121, IWP-427, IWP-953, BAY-60-2770, A-344905, A-350619, A-778935, BI-684067, BI-703704, BAY-41-2272, BAY-41-8543, BAY 60-4552, CF-1571, cinaciguat and HMR-1766.
ii) an NO recoupler, preferably an NO recoupler selected from the group consisting of folic acid and folic acid salts; and
iii) an NO substrate or an NO substrate precursor, preferably an NO substrate or an NO substrate precursor selected from the group consisting of L-Arginine, L-citrulline, salts thereof, hydrates thereof, solvates thereof and combinations thereof.

30. Pharmaceutical composition according to claim 29, wherein:
i) the sGC (positive) modulator is vericiguat; and
iii) the NO substrate or NO substrate precursor is L-citrulline.

31. Method of diagnosing a HFpEF endotype characterised by impaired NO-CGMP-PKG axis functioning in a subject, wherein said method comprises the steps of:
collecting whole blood from the subject, with or without anticoagulant, following which the blood is immediately centrifuged;
collecting plasma;
measuring the NOX5 level in the plasma sample; and
determining that the patient suffers from HFpEF due to impaired NO-cGMP-PKG
axis functioning in case the NOX5 plasma level is at least 105 ng/ML;
and/or wherein said method comprises the steps of:
collecting whole blood, with or without anticoagulant, following which the blood is immediately centrifuged at speeds of 600-800xg for 7.5-15 minutes;
collecting plasma and isolating P-WBC;
cryo-preserving P-WBC by storing at a temperature of −60 to −90° C., using a cryoprotectant, preferably 6% DMSO;
thawing cryopreserved P-WBC;
using the P-WBC to determine the pVASP response (pVASP/VASP) to 1 μM runcaciguat (an sGCa), in the presence of 500 μM IBMX (3-isobutyl-1-methylxanthine) as well as the pVASP response (pVASP/VASP) to 100 μM riociguat (an sGCs), in the presence of 500 μM IBMX; and
determining that the patient suffers from HFpEF due to impaired NO-cGMP-PKG axis functioning in case the sGCa/sGCs ratio, defined as the response to the sGCa divided by the response to the sGCs, is at least 1.05.
